(19) United States
(12) Patent Application Publication
(10) Pub. No.: US 2024/0217981 A1
(43) Pub. Date: Jul. 4, 2024

(54) CRYSTALLINE COMPOUND OF MUSCARINIC ACETYLCHOLINE M1 RECEPTOR ANTAGONISTS

(71) Applicant: Pipeline Therapeutics, Inc., San Diego, CA (US)

(72) Inventors: Jeffrey Roppe, Temecula, CA (US); Jill Melissa Baccei, San Diego, CA (US); Austin Chih-Yu Chen, San Diego, CA (US); Yifeng Xiong, San Diego, CA (US); Thomas Schrader, San Diego, CA (US); Yalda Bravo, San Diego, CA (US)

(21) Appl. No.: 18/286,088

(22) PCT Filed: Apr. 13, 2022
(86) PCT No.: PCT/US2022/024684

§ 371 Date: Oct. 6, 2023

Related U.S. Application Data
(60) Provisional application No. 63/174,415, filed on Apr. 13, 2021.

(51) Int. Cl. C07D 487/08; A61K 31/444; A61K 45/06

(52) U.S. Cl. CPC C07D 487/08; A61K 31/444; A61K 45/06

(57) ABSTRACT

Disclosed herein, inter alia, are a crystalline compound of mAChR M₁ antagonist, its pharmaceutical composition and methods of treatment.

### CROSS REFERENCE TO RELATED APPLICATIONS

This application claims priority to U.S. Provisional Application No. 63/174,415, filed Apr. 13, 2021, which is hereby incorporated by reference in its entirety and for all purposes.

### TECHNICAL FIELD

Disclosed herein, inter alia, are a crystalline compound of mAChR M₁ antagonist, its pharmaceutical composition and methods of treatment.

### BACKGROUND

The human muscarinic acetylcholine receptor M₁ (mAChR M₁) is a protein of 479 amino acids encoded by the CHRM1 gene. The mAChR M₁ is one of five members of the family of muscarinic acetylcholine receptors (M₁-M₅), which are widely expressed throughout the body where they have varying roles in cognitive, sensory, motor, and autonomic functions. The M₁ mAChR is found in both the central and peripheral nervous systems, particularly in the cerebral cortex and sympathetic ganglia. Based on the role of mAChR M₁ in seizure activity and motor control, highly selective mAChR M₁ antagonists may have potential utility in the treatment of some epileptic disorders, as well as certain movement disorders, including Parkinson's disease, dystonia, and fragile X syndrome. Furthermore, mAChR M₁ receptors have been found to be expressed in oligodendrocyte precursor cells (OPCs) which, when activated by their natural ligand acetylcholine, will prevent the differentiation of OPCs into oligodendrocytes. Given the vital role oligodendrocytes play in the remyelination process, highly selective mAChR M₁ antagonists are also expected to have potential utility in the treatment of demyelinating diseases such as multiple sclerosis and diabetic neuropathy.

### SUMMARY

Provided are, inter alia, a crystalline compound, pharmaceutical compositions including the crystalline compound, and methods of treatment using the same.

In an aspect, provided is a crystalline compound having the formula:

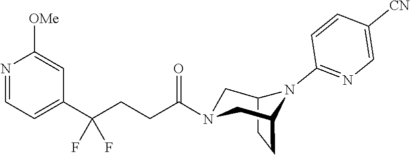

characterized by an x-ray powder diffraction pattern with peaks at 13.4±0.15° 2θ, 14.0±0.15° 2θ, 15.2±0.15° 2θ, 19.3±0.15° 2θ, 20.0±0.15° 2θ and 21.2±0.15° 2θ.

In embodiments, the compound is characterized by an x-ray powder diffraction pattern with peaks at 6.7±0.15° 2θ, at 13.4±0.15° 2θ, 14.0±0.15° 2θ, 15.2±0.15° 2θ, 17.7±0.15° 2θ, 18.1±0.15° 2θ, 18.8±0.15° 2θ, 19.3±0.15° 2θ, 19.8±0.15° 2θ, 20.0±0.15° 2θ, 21.0±0.15° 2θ, 21.2±0.15° 2θ, and 24.1±0.15° 2θ.

In embodiments, the compound is characterized by an x-ray powder diffraction pattern with peaks at 6.7±0.15° 2θ, 10.7±0.15° 2θ, 13.4±0.15° 2θ, 14.0±0.15° 2θ, 15.2±0.15° 2θ, 17.7±0.15° 2θ, 18.1±0.15° 2θ, 18.8±0.15° 2θ, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 27.8±0.15° 2θ, and 30.6±0.15° 2θ.

In embodiments, the compound is characterized by an x-ray powder diffraction pattern with peaks at 6.7±0.15° 2θ, 10.7±0.15° 2θ, 13.4±0.15° 2θ, 14.0±0.15° 2θ, 15.2±0.15° 2θ, 16.6±0.15° 2θ, 17.7±0.15° 2θ, 18.1±0.15° 2θ, 18.4±0.15° 2θ, 18.8±0.15° 2θ, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 26.3±0.15° 2θ, 26.6±0.15° 2θ, 27.0±0.15° 2θ, 27.8±0.15° 2θ, and 30.6±0.15° 2θ.

In embodiments, the compound is further characterized as having a differential scanning calorimetry endotherm onset at a temperature from about 110 to 120° C. In embodiments, the compound is further characterized as having a differential scanning calorimetry endotherm onset at about 117° C.

In an aspect provided is a pharmaceutical composition including a crystalline compound as described herein and at least one pharmaceutically acceptable excipient.

In an aspect provided is a method of treating a neurodegenerative disorder in a subject in need thereof. The method includes administering to the subject a therapeutically effective amount of a crystalline compound described herein.

In embodiments, the method further includes the administration of one or more immunomodulatory agents.

In embodiments, the one or more immunomodulatory agents are selected from: an IFN-β 1 molecule; a corticosteroid; a polymer of glutamic acid, lysine, alanine and tyrosine or glatiramer; an antibody or fragment thereof against alpha-4 integrin or natalizumab; an anthracenedione molecule or mitoxantrone; a fingolimod or FTY720 or other S1P1 functional modulator; a dimethyl fumarate or other NRF2 functional modulator; an antibody to the alpha subunit of the IL-2 receptor of T cells (CD25) or daclizumab; an antibody against CD52 or alemtuzumab; an antibody against CD20 or ocrelizumab; and an inhibitor of a dihydroorotate dehydrogenase or teriflunomide.

In an aspect provided is a method of treating a demyelinating disease in a subject in need thereof. The method includes administering to the subject a therapeutically effective amount of a crystalline compound described herein.

In embodiments, the demyelinating disease is a demyelinating disease of the central nervous system.

In embodiments, the demyelinating disease is multiple sclerosis.

In embodiments, the demyelinating disease is a demyelinating disease of the peripheral nervous system.

In embodiments, the method further includes the administration of one or more immunomodulatory agents.

In embodiments, the one or more immunomodulatory agents are selected from: an IFN-β 1 molecule; a corticosteroid; a polymer of glutamic acid, lysine, alanine and tyrosine or glatiramer; an antibody or fragment thereof against alpha-4 integrin or natalizumab; an anthracenedione molecule or mitoxantrone; a fingolimod or FTY720 or other S1P1 functional modulator; a dimethyl fumarate or other NRF2 functional modulator; an antibody to the alpha subunit of the IL-2 receptor of T cells (CD25) or daclizumab; an antibody against CD52 or alemtuzumab; an antibody against CD20 or ocrelizumab; and an inhibitor of a dihydroorotate dehydrogenase or teriflunomide.

In an aspect provided is a method of treating a neuropathic disease, optionally a peripheral neuropathy, in a subject in need thereof, comprising administering to the subject a therapeutically effective amount of a crystalline compound described herein.

In embodiments, the neuropathic disease is diabetic neuropathy.

In embodiments, the method further includes the administration of one or more immunomodulatory agents.

In embodiments, the one or more immunomodulatory agents are selected from: an IFN-β 1 molecule; a corticosteroid; a polymer of glutamic acid, lysine, alanine and tyrosine or glatiramer; an antibody or fragment thereof against alpha-4 integrin or natalizumab; an anthracenedione molecule or mitoxantrone; a fingolimod or FTY720 or other S1P1 functional modulator; a dimethyl fumarate or other NRF2 functional modulator; an antibody to the alpha subunit of the IL-2 receptor of T cells (CD25) or daclizumab; an antibody against CD52 or alemtuzumab; an antibody against CD20 or ocrelizumab; and an inhibitor of a dihydroorotate dehydrogenase or teriflunomide.

In an aspect provided is a method of modulating muscarinic acetylcholine receptor M₁ activity in a subject comprising administering to the subject a crystalline compound as described herein.

In embodiments, the compound acts as a selective M₁ antagonist.

Other aspects of the invention are disclosed infra.

### BRIEF DESCRIPTION OF THE DRAWINGS

FIG. 1 shows XRPD pattern of the crystalline 6-((1R,5S)-3-(4,4-difluoro-4-(2-methoxypyridin-4-yl)butanoyl)-3,8-diazabicyclo [3.2.1]octan-8-yl)nicotinonitrile.

FIG. 2A shows TGA/DSC curves of the crystalline compound.

FIG. 2B shows cycle-DSC curve of the crystalline compound.

FIG. 2C shows XRPD overlay of the crystalline compound before and after heating.

FIG. 2D shows HPLC chromatogram of the crystalline compound.

FIG. 2E shows PLM image of the crystalline compound.

FIG. 3A shows PLM images of single crystals obtained in polymorph screening of the compound.

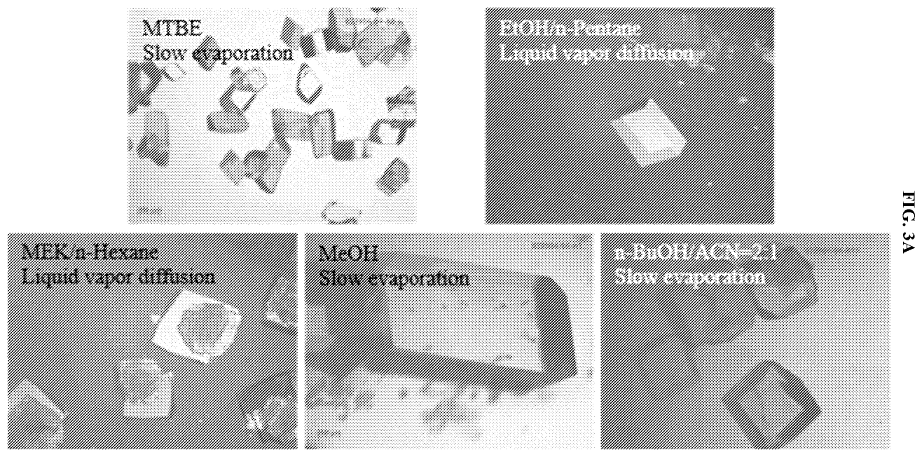

FIG. 3B shows XRPD overlay of single crystal samples.

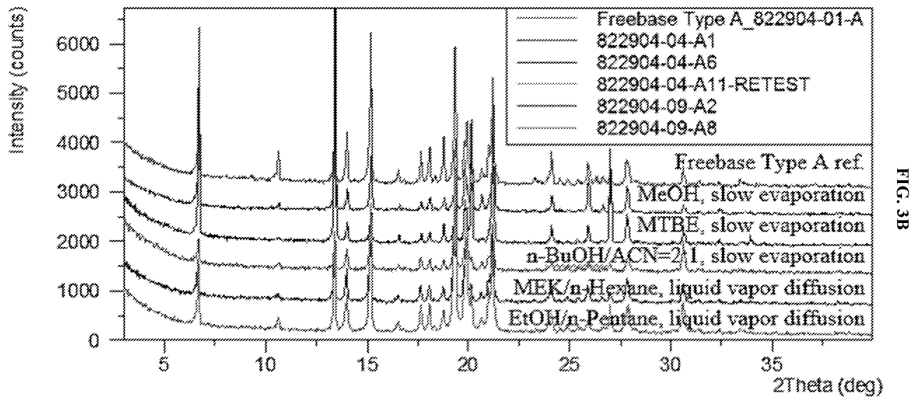

FIG. 4A shows diagram of kinetic solubility evaluation of the crystalline compound in SGF, FeSSIF, FaSSIF, and water.

FIG. 4B shows XRPD overlay of the crystalline compound in SGF and H₂O.

FIG. 4C shows XRPD overlay of the crystalline compound in FaSSIF and FeSSIF.

FIG. 4D shows HPLC chromatogram overlay of the crystalline compound before and after stability study.

FIG. 4E shows XRPD overlay of the crystalline compound before and after stability study.

FIG. 4F shows DVS plot of the crystalline compound.

FIG. 4G shows XRPD overlay of the crystalline compound before and after DVS.

FIG. 5A shows XRPD overlay of various recrystallized samples.

FIG. 5B shows XRPD overlay of the crystalline compound before and after grinding experiments.

### DETAILED DESCRIPTION

### Definitions

The abbreviations used herein have their conventional meaning within the chemical and biological arts. The chemical structures and formulae set forth herein are constructed according to the standard rules of chemical valency known in the chemical arts.

As used herein, the term “about” means a range of values including the specified value, which a person of ordinary skill in the art would consider reasonably similar to the specified value. In embodiments, “about” means within a standard deviation using measurements generally acceptable in the art. In embodiments, “about” means a range extending to +/−10% of the specified value. In embodiments, “about” includes the specified value (e.g., ±0.10, ±0.15, ±0.20, or ±0.25).

The term “polymorph” is used in accordance with its ordinary meaning and refers to a crystalline form of a compound.

As used herein, the term “crystalline” or “crystalline state” or “crystalline form” means having a physical state that is a regular three-dimensional array of atoms, ions, molecules, or molecular assemblies. Crystalline states have lattice arrays of building blocks that are arranged according to well-defined symmetries into unit cells that are repeated in three dimensions. In contrast, the term “amorphous” or “amorphous state” or “amorphous form” refers to a non-crystalline solid state. The physical state of a compound may be determined by techniques such as X-ray powder diffraction, polarized light microscopy and/or differential scanning calorimetry.

A compound, salt form, crystal polymorph, therapeutic agent, or other composition described herein may be referred to as being characterized by graphical data “substantially as depicted in” a figure. Such data may include, but is not limited to, X-ray powder diffraction spectra, NMR spectra, differential scanning calorimetry curves, and thermogravimetric analysis curves, among others. As is known in the art, such graphical data may provide additional technical information to further define the compound, salt form, crystal polymorph, therapeutic agent, or other composition. As is understood by one skilled in the art, such graphical representations of data may be subject to small variation, e.g., in peak relative intensities and peak positions due to factors such as variations in instrument response and variations in sample concentration and purity.

Where substituent groups are specified by their conventional chemical formulae, written from left to right, they equally encompass the chemically identical substituents that would result from writing the structure from right to left, e.g., —CH₂O—is equivalent to —OCH₂—.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” is used according to its plain and ordinary meeting and includes oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation). The compositions of the present invention can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995); as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

The terms “allosteric site” and “allosteric binding site” refer to a ligand binding site that is topographically distinct from the orthosteric binding site.

The terms “orthosteric site” and “orthosteric binding site” refer to the primary binding site on a receptor that is recognized by an endogenous ligand or agonist for the receptor. For example, the orthosteric site on the muscarinic acetylcholine M₁ receptor is the site that acetylcholine binds.

The term “ligand” refers to a natural or synthetic molecule that is capable of binding to or associating with a receptor to form a complex and mediate, prevent, or modify a biological effect. The term “ligand” is meant to encompass allosteric modulators, inhibitors, activators, agonists, antagonists, natural substrates, and analogs of natural substrates.

The terms “natural ligand” and “endogenous ligand” refer to a naturally occurring ligand which binds to a receptor.

In embodiments, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

In embodiments, the terms “inhibitor”, “repressor”, “antagonist”, or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

In embodiments, the term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “mAChR M₁ receptor antagonist” refers to any exogenously administered compound or agent that is capable of partially or completely inhibiting, or reversing, the effect of an agonist (e.g. acetylcholine) on the mAChR M₁ receptor. The term is inclusive of compounds or agents characterized or described as antagonists, partial antagonists, and negative allosteric modulators. For example, mAChR M₁ receptor antagonists can mediate their effects by binding to the orthosteric site or to allosteric sites, or they may interact at unique binding sites not normally involved in the biological regulation of the receptor's activity. Thus, a mAChR M₁ receptor antagonist directly or indirectly inhibits the activity of the mAChR M₁ receptor in the presence or in the absence of acetylcholine, or another agonist, in an animal, in particular a mammal, for example a human. In various aspects, a mAChR M₁ receptor antagonist decreases the activity of the mAChR M₁ receptor in a cell in the presence of extracellular acetylcholine. In some embodiments, a compound that is a “mAChR M₁ receptor antagonist” includes a compound that is a “mAChR M₁ receptor competitive antagonist,” a “mAChR M₁ receptor noncompetitive antagonist,” a “mAChR M₁ receptor partial antagonist,” or a “mAChR M₁ receptor negative allosteric modulator.”

The term “mAChR M₁ receptor competitive antagonist” refers to any exogenously administered compound or agent that is capable of binding to the orthosteric site of mAChR M₁ receptors without activating the receptor. Thus, a competitive antagonist can interact with a mAChR M₁ receptor and compete with the endogenous ligand, acetylcholine, for binding to the receptor and decrease the ability of the receptor to transduce an intracellular signal in response to endogenous ligand binding.

The term “mAChR M₁ receptor noncompetitive antagonist” refers to any exogenously administered compound or agent that binds to site that is not the orthosteric binding site of mAChR M₁ receptors, and is capable of partially or completely inhibiting, or reversing, the effect of an agonist (e.g. acetylcholine) on the mAChR M₁ receptor. Thus, a non-competitive antagonist can interact with a mAChR M₁ receptor and decrease the binding of the endogenous ligand, acetylcholine, to the receptor and/or decrease the ability of the receptor to transduce an intracellular signal in response to endogenous ligand binding.

The term “mAChR M₁ partial antagonist” refers to any exogenously administered compound or agent that can bind to an orthosteric or an allosteric site, but the effect of binding is to only partially block effect of mAChR M₁ receptor response to an agonist, e.g. acetylcholine. Thus, a partial antagonist can interact with a mAChR M₁ receptor and but is not capable of fully inhibiting the response of the mAChR M₁ receptor to an agonist, e.g. acetylcholine.

The term “mAChR M₁ negative allosteric modulator” refers to any exogenously administered compound or agent that binds an allosteric site that directly or indirectly inhibits the activity of the mAChR M₁ receptor in the presence of acetylcholine, or another agonist, in an animal, in particular a mammal, for example a human. For example, while not intended to be limiting towards the present disclosure, a selective muscarinic M₁ negative allosteric modulator can preferentially bind to the muscarinic M₁ receptor and decrease muscarinic M₁ signaling by acting as a non-competitive antagonist. In one aspect, a mAChR M₁ receptor negative allosteric modulator decreases the activity of the mAChR M₁ receptor in a cell in the presence of extracellular acetylcholine.

In embodiments, “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward muscarinic acetylcholine M₁ receptor (mAChR M₁) would preferentially inhibit mAChR M₁ over other muscarinic receptors). In embodiments, a “muscarinic acetylcholine M₁ receptor selective compound” or “mAChR M₁-selective compound” refers to a compound (e.g., compounds described herein) having selectivity towards muscarinic acetylcholine M₁ receptor (mAChR M₁). In embodiments, the compound (e.g., compound described herein) is about 5-fold, 10-fold, 20-fold, 30-fold, 40-fold, 50-fold, or about 100-fold more selective for muscarinic acetylcholine M₁ receptor (mAChR M₁) over one or more of the mAChR M₂, M₃, M₄, or M₅ receptors. In embodiments, the compound (e.g., compound described herein) is at least 5-fold, 10-fold, 20-fold, 30-fold, 40-fold, 50-fold, or at least 100-fold more selective for muscarinic acetylcholine M₁ receptor (mAChR M₁) over one or more of the mAChR M₂, M₃, M₄, or M₅ receptors.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the subject is a human.

As used herein, “treatment” or “treating” or “palliating” or “ameliorating” are used interchangeably herein. These terms refer to an approach for obtaining beneficial or desired results including but not limited to therapeutic benefit and/or prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

As used herein, “EC₅₀” is intended to refer to the concentration of a substance (e.g., a compound or a drug) that is required for 50% activation or enhancement of a biological process, or component of a process, including a protein, subunit, organelle, ribonucleoprotein, etc. For example, EC₅₀ can refer to the concentration of agonist that provokes a response halfway between the baseline and maximum response in an in vitro assay. In some embodiments, in vitro assay systems utilize a cell line that either expresses endogenously a target of interest or has been transfected with a suitable expression vector that directs expression of a recombinant form of the target.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. For example, IC₅₀ refers to the half maximal (50%) inhibitory concentration (IC) of a substance as determined in a suitable assay. For example, an IC₅₀ for mAChR M₁ receptor can be determined in an in vitro assay system.

### Compounds

Provided herein is a crystalline compound of 6-(3-(4,4-difluoro-4-(2-methoxypyridin-4-yl)butanoyl)-3,8-diazabicyclo[3.2.1]octan-8-yl)nicotinonitrile (“compound”) having the structure:

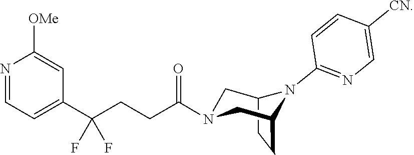

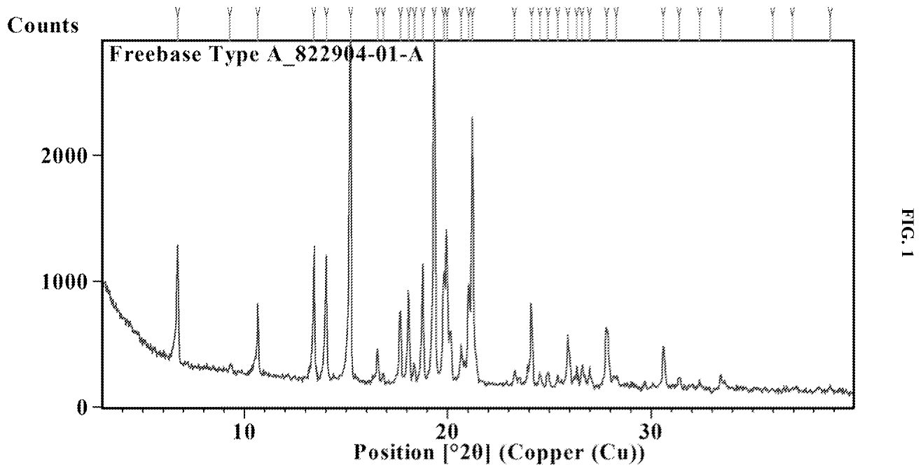

Crystalline forms of the compound may be characterized by the Cu K-α x-ray powder diffraction (XRPD) pattern substantially as shown in FIG. 1.

In an aspect, provided is a crystalline compound having the formula:

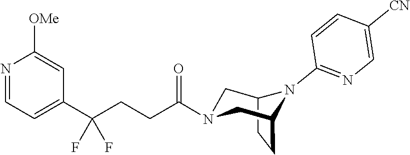

characterized by an x-ray powder diffraction pattern with peaks at 13.4±0.15° 2θ, 14.0±0.15° 2θ, 15.2±0.15° 2θ, 19.3±0.15° 2θ, 20.0±0.15° 20 and 21.2±0.15° 2θ.

In embodiments, the compound is further characterized by an x-ray powder diffraction pattern with peaks at 6.7±0.15° 2θ, at 13.4±0.15° 2θ, 14.0±0.15° 2θ, 15.2 0.15° 2θ, 17.7±0.15° 2θ, 18.1±0.15° 2θ, 18.8±0.15° 2θ, 19.3±0.15° 2θ, 19.8±0.15° 2θ, 20.0±0.15° 2θ, 21.0±0.15° 2θ, 21.2±0.15° 2θ, and 24.1±0.15° 2θ.

In embodiments, the compound is further characterized by an x-ray powder diffraction pattern with peaks at 6.7±0.15° 2θ, 10.7±0.15° 2θ, 13.4±0.15° 2θ, 14.0±0.15° 20, 15.2±0.15° 2θ, 17.7±0.15° 2θ, 18.1±0.15° 2θ, 18.8±0.15° 2θ, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 27.8±0.15° 2θ, and 30.6±0.15° 2θ.

In embodiments, the compound is further characterized by an x-ray powder diffraction pattern with peaks at 6.7±0.15° 2θ, 10.7±0.15° 2θ, 13.4±0.15° 2θ, 14.0±0.15° 20, 15.2±0.15° 2θ, 16.6±0.15° 2θ, 17.7±0.15° 2θ, 18.1±0.15° 2θ, 18.4±0.15° 2θ, 18.8±0.15° 2θ, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 26.3±0.15° 2θ, 26.6±0.15° 2θ, 27.0±0.15° 2θ, 27.8±0.15° 2θ, and 30.6±0.15° 2θ.

Alternatively, the crystalline compound may be characterized by the x-ray powder diffraction pattern with peaks at:
i) about 13.4° 2θ, about 14.0° 2θ, about 15.2° 2θ, about 19.3° 2θ, about 20.0° 2θ and about 21.2° 2θ;
ii) about 6.7° 2θ, about 13.4° 2θ, about 14.0° 2θ, about 15.2° 2θ, about 17.7° 2θ, about 18.1° 2θ, about 18.8° 2θ, about 19.3° 2θ, about 19.8° 2θ, about 20.0° 2θ, about 21.0° 2θ, about 21.2° 2θ, and about 24.1° 2θ;
iii) about 6.7° 2θ, about 10.7° 2θ, about 13.4° 2θ, about 14.0° 2θ, about 15.2° 2θ, about 17.7° 2θ, about 18.1° 2θ, about 18.8° 2θ, about 19.3° 2θ, about 19.8° 2θ, about 20.0° 2θ, about 20.7° 20, about 21.0° 2θ, about 21.2° 2θ, about 24.1° 2θ, about 25.9° 2θ, about 27.8° 2θ, and about 30.6° 2θ; or
iv) about 6.7° 2θ, about 10.7° 2θ, about 13.4° 2θ, about 14.0° 2θ, about 15.2° 2θ, about 16.6° 2θ, about 17.7° 2θ, about 18.1° 2θ, about 18.4° 2θ, about 18.8° 2θ, about 19.3° 2θ, about 19.8° 2θ, about 20.0° 2θ, about 20.7° 2θ, about 21.0° 2θ, about 21.2° 2θ, about 24.1° 2θ, about 25.9° 2θ, about 26.3° 2θ, about 26.6° 2θ, about 27.0° 2θ, about 27.8° 2θ, and about 30.6° 2θ.

Alternatively, the crystalline compound may be characterized by the x-ray powder diffraction pattern with peaks at:
i) about 13.4° 2θ, about 14.0° 2θ, about 15.2° 2θ, about 19.3° 2θ, about 20.0° 2θ and about 21.2° 2θ;
ii) about 6.7° 2θ, about 13.4° 2θ, about 14.0° 2θ, about 15.2° 2θ, about 17.7° 2θ, about 18.1° 2θ, about 18.8° 2θ, about 19.3° 2θ, about 19.8° 2θ, about 20.0° 2θ, about 21.0° 2θ, about 21.2° 2θ, and about 24.1° 2θ;
iii) about 6.7° 2θ, about 10.7° 2θ, about 13.4° 2θ, about 14.0° 2θ, about 15.2° 2θ, about 17.7° 2θ, about 18.1° 2θ, about 18.8° 2θ, about 19.3° 2θ, about 19.8° 2θ, about 20.0° 2θ, about 20.7° 2θ, about 21.0° 2θ, about 21.2° 2θ, about 24.1° 2θ, about 25.9° 2θ, about 27.8° 2θ, and about 30.6° 2θ; and/or
iv) about 6.7° 2θ, about 10.7° 2θ, about 13.4° 2θ, about 14.0° 2θ, about 15.2° 2θ, about 16.6° 2θ, about 17.7° 2θ, about 18.1° 2θ, about 18.4° 2θ, about 18.8° 2θ, about 19.3° 2θ, about 19.8° 2θ, about 20.0° 2θ, about 20.7° 2θ, about 21.0° 2θ, about 21.2° 2θ, about 24.1° 2θ, about 25.9° 2θ, about 26.3° 2θ, about 26.6° 2θ, about 27.0° 2θ, about 27.8° 2θ, and about 30.6° 2θ.

In embodiments, the crystalline compound may be characterized by the x-ray powder diffraction pattern with peaks at:
i) 13.4±0.15° 2θ, 14.0±0.15° 2θ, 15.2±0.15° 2θ, 19.3±0.15° 2θ, 20.0±0.15° 20 and 21.2±0.15° 2θ;
ii) 6.7±0.15° 2θ, at 13.4±0.15° 2θ, 14.0±0.15° 2θ, 15.2±0.15° 2θ, 17.7±0.15° 2θ, 18.1±0.15° 2θ, 18.8±0.15° 2θ, 19.3±0.15° 2θ, 19.8±0.15° 2θ, 20.0±0.15° 2θ, 21.0±0.15° 2θ, 21.2±0.15° 2θ, and 24.1±0.15° 2θ;
iii) 6.7±0.15° 2θ, 10.7±0.15° 2θ, 13.4±0.15° 2θ, 14.0±0.15° 2θ, 15.2±0.15° 2θ, 17.7±0.15° 2θ, 18.1±0.15° 2θ, 18.8±0.15° 2θ, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 27.8±0.15° 2θ, and 30.6±0.15° 2θ; or
iv) 6.7±0.15° 2θ, 10.7±0.15° 2θ, 13.4±0.15° 2θ, 14.0±0.15° 2θ, 15.2±0.15° 2θ, 16.6±0.15° 2θ, 17.7±0.15° 2θ, 18.1±0.15° 2θ, 18.4±0.15° 2θ, 18.8±0.15° 2θ, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 26.3±0.15° 2θ, 26.6±0.15° 2θ, 27.0±0.15° 2θ, 27.8±0.15° 2θ, and 30.6±0.15° 2θ.

In embodiments, the crystalline compound may be characterized by the x-ray powder diffraction pattern with peaks at:
i) 13.4±0.15° 2θ, 14.0±0.15° 2θ, 15.2±0.15° 2θ, 19.3±0.15° 2θ, 20.0±0.15° 20 and 21.2±0.15° 2θ;
ii) 6.7±0.15° 2θ, at 13.4±0.15° 2θ, 14.0±0.15° 2θ, 15.2±0.15° 2θ, 17.7±0.15° 20, 18.1±0.15° 2θ, 18.8±0.15° 2θ, 19.3±0.15° 2θ, 19.8±0.15° 2θ, 20.0±0.15° 2θ, 21.0±0.15° 2θ, 21.2±0.15° 2θ, and 24.1±0.15° 2θ;
iii) 6.7±0.15° 2θ, 10.7±0.15° 2θ, 13.4±0.15° 2θ, 14.0±0.15° 2θ, 15.2±0.15° 2θ, 17.7±0.15° 2θ, 18.1±0.15° 2θ, 18.8±0.15° 2θ, 19.3±0.15° 2θ, 19.8±0.15° 20, 20.0±0.15° 2θ, 20.7±0.15° 2θ, 21.0±0.15° 2θ, 21.2±0.15° 2θ, 24.1±0.15° 2θ, 25.9±0.15° 2θ, 27.8±0.15° 2θ, and 30.6±0.15° 2θ; and/or
iv) 6.7±0.15° 2θ, 10.7±0.15° 2θ, 13.4±0.15° 2θ, 14.0±0.15° 2θ, 15.2±0.15° 2θ, 16.6±0.15° 2θ, 17.7±0.15° 2θ, 18.1±0.15° 2θ, 18.4±0.15° 2θ, 18.8±0.15° 20, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 26.3±0.15° 2θ, 26.6±0.15° 2θ, 27.0±0.15° 2θ, 27.8±0.15° 2θ, and 30.6±0.15° 2θ.

The peaks of x-ray powder diffraction pattern of the crystalline compound are provided in Table 1.

TABLE 1
XRPD peak list of the crystalline compound

| | | FWHM | | Rel. |
|---|---|---|---|---|
| Pos. | Height | Left | d-spacing | Int. |
| [°2θ] | [cts] | [°2θ] | [Å] | [%] |
| 6.70 | 915.34 | 0.0768 | 13.19 | 32.51 |
| 9.28 | 38.38 | 0.1535 | 9.53 | 1.36 |
| 10.65 | 555.89 | 0.0768 | 8.30 | 19.74 |
| 13.41 | 1062.78 | 0.1023 | 6.60 | 37.75 |
| 14.01 | 985.85 | 0.1023 | 6.32 | 35.01 |
| 15.20 | 2815.56 | 0.0768 | 5.83 | 100.00 |
| 16.55 | 265.93 | 0.0768 | 5.36 | 9.45 |
| 16.83 | 72.60 | 0.0768 | 5.27 | 2.58 |
| 17.67 | 587.97 | 0.0768 | 5.02 | 20.88 |
| 18.08 | 732.07 | 0.1023 | 4.91 | 26.00 |
| 18.37 | 147.02 | 0.1023 | 4.83 | 5.22 |
| 18.78 | 882.86 | 0.1023 | 4.72 | 31.36 |
| 19.34 | 2711.97 | 0.1023 | 4.59 | 96.32 |
| 19.80 | 844.20 | 0.0768 | 4.48 | 29.98 |
| 19.96 | 1136.38 | 0.0768 | 4.45 | 40.36 |
| 20.66 | 309.00 | 0.1023 | 4.30 | 10.97 |
| 21.02 | 764.15 | 0.0768 | 4.23 | 27.14 |
| 21.22 | 2049.47 | 0.0768 | 4.19 | 72.79 |
| 23.29 | 93.48 | 0.1279 | 3.82 | 3.32 |
| 24.12 | 616.64 | 0.1023 | 3.69 | 21.90 |
| 24.54 | 96.36 | 0.1023 | 3.63 | 3.42 |
| 24.92 | 100.67 | 0.1279 | 3.57 | 3.58 |
| 25.40 | 86.28 | 0.0768 | 3.51 | 3.06 |
| 25.91 | 407.56 | 0.1023 | 3.44 | 14.48 |
| 26.33 | 161.32 | 0.0768 | 3.38 | 5.73 |
| 26.61 | 155.95 | 0.0768 | 3.35 | 5.54 |
| 26.97 | 141.47 | 0.1023 | 3.31 | 5.02 |
| 27.81 | 473.43 | 0.1535 | 3.21 | 16.81 |
| 28.27 | 78.67 | 0.2047 | 3.16 | 2.79 |
| 30.61 | 342.97 | 0.1023 | 2.92 | 12.18 |
| 31.38 | 86.51 | 0.1535 | 2.85 | 3.07 |
| 32.39 | 60.16 | 0.1535 | 2.76 | 2.14 |
| 33.42 | 119.77 | 0.0768 | 2.68 | 4.25 |
| 35.96 | 17.96 | 0.1791 | 2.50 | 0.64 |
| 36.94 | 17.17 | 0.6140 | 2.43 | 0.61 |
| 38.82 | 41.63 | 0.1535 | 2.32 | 1.48 |

In embodiments, the x-ray powder diffraction pattern of the crystalline compound has one or more of the peaks set forth in Table 1.

In embodiments, the compound is further characterized as having a differential scanning calorimetry endotherm onset at about 117° C.

### Methods of Producing Crystalline Compound

Provided herein are methods of producing the crystalline compound described herein.

In an aspect, a method of producing a crystalline compound include steps of:
i) preparing an admixture comprising a solvent component and a compound having a structure of

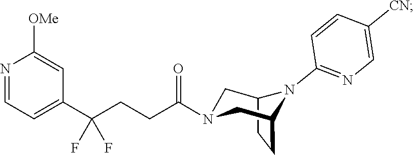

ii) adding an anti-solvent component to the admixture; and
iii) obtaining the crystalline compound.

In embodiments, the solvent component includes one or more selected from the group consisting of acetonitrile, tetrahydrofuran, dimethylsulfoxide, methanol, isopropyl acetate, anisole, methyl isobutyl ketone, dichloromethane, toluene, acetone, ethyl acetate, and chloroform. In embodiments, the solvent component is acetonitrile. In embodiments, the solvent component is methanol. In embodiments, the solvent component is tetrahydrofuran. In embodiments, the solvent component is dimethylsulfoxide. In embodiments, the solvent component is isopropyl acetate. In embodiments, the solvent component is anisole. In embodiments, the solvent component is methyl isobutyl ketone. In embodiments, the solvent component is dichloromethane. In embodiments, the solvent component is toluene. In embodiments, the solvent component is acetone. In embodiments, the solvent component is ethyl acetate. In embodiments, the solvent component is chloroform.

In embodiments, the anti-solvent component includes one or more selected from the group consisting of water, n-hexane, n-heptane, and cyclohexane. In embodiments, the anti-solvent component is water. In embodiments, the anti-solvent component is n-hexane. In embodiments, the anti-solvent component is n-heptane. In embodiments, the anti-solvent component is cyclohexane.

In embodiments, the method further includes, after step (ii), evaporating the solvent component and the anti-solvent component.

In an aspect, a method of producing a crystalline compound includes steps of:
(i) preparing an admixture including a solvent component and a compound having a structure of

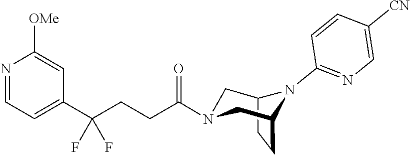

in a container; and
(ii) evaporating the solvent component at room temperature to obtain the crystalline compound.

In embodiments, the solvent component includes one or more selected from the group consisting of methanol, dichloromethane, acetone, acetonitrile, tetrahydrofuran, methyl tert-butyl ether, ethyl acetate, methyl ethyl ketone, water, cyclohexane, n-butyl alcohol, isopropyl alcohol, and 2-methyltetrahydrofuran. In embodiments, the solvent component is methanol. In embodiments, the solvent component is dichloromethane. In embodiments, the solvent component is acetone. In embodiments, the solvent component is acetonitrile. In embodiments, the solvent component is tetrahydrofuran. In embodiments, the solvent component is methyl tert-butyl ether. In embodiments, the solvent component is ethyl acetate. In embodiments, the solvent component is methyl ethyl ketone. In embodiments, the solvent component is water. In embodiments, the solvent component is cyclohexane. In embodiments, the solvent component is n-butyl alcohol. In embodiments, the solvent component is isopropyl alcohol. In embodiments, the solvent component is 2-methyltetrahydrofuran.

In an aspect, a method of producing a crystalline compound includes steps of.
(i) preparing an admixture comprising a solvent component and a compound having a structure of

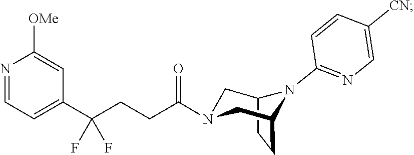

(ii) heating the admixture to a first temperature;
(iii) cooling the admixture to a second temperature;
(iv) repeating the steps (ii) and (iii); and
(v) obtaining the crystalline compound.

In embodiments, the first temperature ranges from about 40° C. to about 60° C.

In embodiments, the second temperature ranges from about 0° C. to about 10° C.

In embodiments, the steps (ii) and (iii) are repeated three times.

In embodiments, the method further includes, after step (iv), cooling the admixture further to −20° C. and then allowing the slow evaporation of the solvent component at room temperature.

In embodiments, the solvent component includes one or more selected from the group consisting of ethanol, isopropyl alcohol, methyl tert-butyl ether, cyclopentyl methyl ether, n-propanol, water, methyl ethyl ketone, n-butyl alcohol, ethyl acetate, cyclohexane, acetone, n-hexane, n-heptane, methyl isobutyl ketone, anisole, isopropyl acetate, and acetonitrile. In embodiments, the solvent component includes ethanol. In embodiments, the solvent component includes isopropyl alcohol. In embodiments, the solvent component includes methyl tert-butyl ether. In embodiments, the solvent component includes cyclopentyl methyl ether. In embodiments, the solvent component includes n-propanol. In embodiments, the solvent component includes water. In embodiments, the solvent component includes methyl ethyl ketone. In embodiments, the solvent component includes n-butyl alcohol. In embodiments, the solvent component includes ethyl acetate. In embodiments, the solvent component includes cyclohexane. In embodiments, the solvent component includes acetone. In embodiments, the solvent component includes n-hexane. In embodiments, the solvent component includes n-heptane. In embodiments, the solvent component includes methyl isobutyl ketone. In embodiments, the solvent component includes anisole. In embodiments, the solvent component includes isopropyl acetate. In embodiments, the solvent component includes acetonitrile.

In an aspect, a method of producing a crystalline compound includes steps of.
(i) preparing an admixture comprising a solvent component and a compound having a structure of

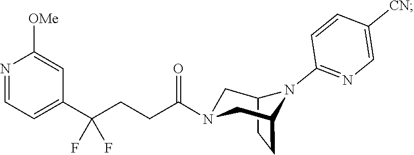

and
(ii) stirring the admixture to obtain the crystalline compound.

In embodiments, the stirring the admixture is performed at a speed about 750 rpm at room temperature for at least one week.

In embodiments, the method may further include, after the step (ii), cooling the admixture to a temperature from about −20° C. to about 5° C.

In embodiments, the stirring in the step (ii) is performed at a temperature from about 40° C. to about 60° C.

In embodiments, the method may further include, after the step (ii), cooling the admixture to a temperature from about −20° C. to about 5° C.

In embodiments, the solvent component is evaporated to obtain the crystalline compound.

In embodiments, the solvent component includes one or more selected from the group consisting of ethanol, isopropyl alcohol, methyl tert-butyl ether, cyclopentyl methyl ether, dimethoxyethane, methyl isobutyl ketone, isopropyl acetate, acetonitrile, m-xylene, n-hexane, n-heptane, methyl acetate, ethyl acetate, dimethylsulfoxide, water, methyl ethyl ketone, 1,4-dioxane, toluene, 2-methyltetrahydrofuran, cyclohexane, dichloromethane, acetone, and chloroform. In embodiments, the solvent component includes ethanol. In embodiments, the solvent component includes isopropyl alcohol. In embodiments, the solvent component includes methyl tert-butyl ether. In embodiments, the solvent component includes cyclopentyl methyl ether. In embodiments, the solvent component includes dimethoxyethane. In embodiments, the solvent component includes methyl isobutyl ketone. In embodiments, the solvent component includes isopropyl acetate. In embodiments, the solvent component includes acetonitrile. In embodiments, the solvent component includes m-xylene. In embodiments, the solvent component includes n-hexane. In embodiments, the solvent component includes n-heptane. In embodiments, the solvent component includes methyl acetate. In embodiments, the solvent component includes ethyl acetate. In embodiments, the solvent component includes dimethylsulfoxide. In embodiments, the solvent component includes water. In embodiments, the solvent component includes methyl ethyl ketone. In embodiments, the solvent component includes 1,4-dioxane. In embodiments, the solvent component includes toluene. In embodiments, the solvent component includes 2-methyltetrahydrofuran. In embodiments, the solvent component includes cyclohexane. In embodiments, the solvent component includes dichloromethane. In embodiments, the solvent component includes acetone. In embodiments, the solvent component includes chloroform.

In an aspect, a method of producing a crystalline compound includes steps of.
(i) placing a compound having a structure of

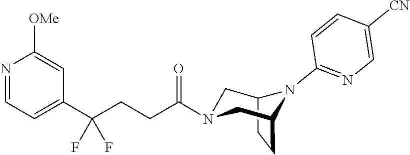

in an unsealed first container;
(ii) placing the unsealed first container containing the compound in a second container containing a vaporizing solvent;
(iii) sealing the second container; and
(iv) obtaining the crystalline compound.

In embodiments, the second container is kept at room temperature for at least two weeks.

In embodiments, the vaporizing solvent includes one or more selected from the group consisting of water, dichloromethane, chloroform, methanol, ethanol, acetonitrile, acetone, ethyl acetate, methyl tert-butyl ether, methyl ethyl ketone, tetrahydrofuran, and dimethylsulfoxide. In embodiments, the vaporizing solvent includes water. In embodiments, the vaporizing solvent includes dichloromethane. In embodiments, the vaporizing solvent includes chloroform. In embodiments, the vaporizing solvent includes methanol. In embodiments, the vaporizing solvent includes ethanol. In embodiments, the vaporizing solvent includes acetonitrile. In embodiments, the vaporizing solvent includes acetone. In embodiments, the vaporizing solvent includes ethyl acetate. In embodiments, the vaporizing solvent includes methyl tert-butyl ether. In embodiments, the vaporizing solvent includes methyl ethyl ketone. In embodiments, the vaporizing solvent includes tetrahydrofuran. In embodiments, the vaporizing solvent includes dimethylsulfoxide.

In an aspect, a method of producing a crystalline compound includes steps of.
(i) preparing an admixture comprising a solvent component and a compound having a structure of

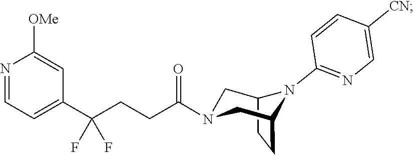

(ii) placing the admixture in an unsealed first container;
(iii) placing the unsealed first container containing the compound and a solvent component in a second container containing an anti-solvent component,
(iv) sealing the second container; and
(v) obtaining the crystalline compound.

In embodiments, the second container is kept at room temperature for at least two weeks.

In embodiments, the solvent component includes one or more selected from the group consisting of 2-methyltetrahydrofuran, methyl ethyl ketone, ethyl acetate, 1,4-dioxane, toluene, isopropyl acetate, anisole, ethanol, and chloroform. In embodiments, the solvent component includes 2-methyltetrahydrofuran. In embodiments, the solvent component includes methyl ethyl ketone. In embodiments, the solvent component includes ethyl acetate. In embodiments, the solvent component includes 1,4-dioxane. In embodiments, the solvent component includes toluene. In embodiments, the solvent component includes isopropyl acetate. In embodiments, the solvent component includes anisole. In embodiments, the solvent component includes ethanol. In embodiments, the solvent component includes chloroform.

In embodiments, the anti-solvent component includes one or more selected from n-hexane, water, cyclohexane, and n-pentane.

In an aspect, a method of producing a crystalline compound includes steps of:
(i) preparing an admixture comprising a solvent component and a compound having a structure of

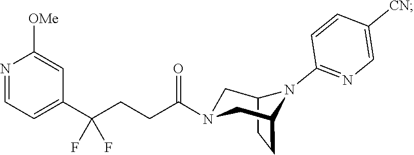

(ii) transferring the admixture into a container containing a polymer mixture; and
(iii) obtaining the crystalline compound.

In embodiments, the polymer mixture is a mixture of polyvinyl pyrrolidone (PVP), polyvinyl alcohol (PVA), polyvinylchloride (PVC), polyvinyl acetate (PVAC), hypromellose (HPMC), and methyl cellulose (MC) in a mass ratio of 1:1:1:1:1:1, or a mixture of polycaprolactone (PCL), polyethylene glycol (PEG), poly (methyl methacrylate) (PMMA), sodium alginate (SA), and hydroxyethyl cellulose (HEC) in a mass ratio of 1:1:1:1:1. In embodiments, the polymer mixture is a mixture of polyvinyl pyrrolidone (PVP), polyvinyl alcohol (PVA), polyvinylchloride (PVC), polyvinyl acetate (PVAC), hypromellose (HPMC), and methyl cellulose (MC) in a mass ratio of 1:1:1:1:1:1. In embodiments, the polymer mixture is a mixture of polycaprolactone (PCL), polyethylene glycol (PEG), poly (methyl methacrylate) (PMMA), sodium alginate (SA), and hydroxyethyl cellulose (HEC) in a mass ratio of 1:1:1:1:1.

In embodiments, the method may further include, after step (ii), evaporating the solvent component.

In embodiments, the solvent component includes one or more selected from the group consisting of isopropyl acetate, water, acetonitrile, ethanol, chloroform, n-hexane, acetone, methyl ethyl ketone, methyl tert-butyl ether, dichloromethane, and 1,4-dioxane. In embodiments, the solvent component includes isopropyl acetate. In embodiments, the solvent component includes water. In embodiments, the solvent component includes acetonitrile. In embodiments, the solvent component includes ethanol. In embodiments, the solvent component includes chloroform. In embodiments, the solvent component includes n-hexane. In embodiments, the solvent component includes acetone. In embodiments, the solvent component includes methyl ethyl ketone. In embodiments, the solvent component includes methyl tert-butyl ether. In embodiments, the solvent component includes dichloromethane. In embodiments, the solvent component includes 1,4-dioxane.

In an aspect, a method of producing a crystalline compound includes a step of milling a compound having a structure of

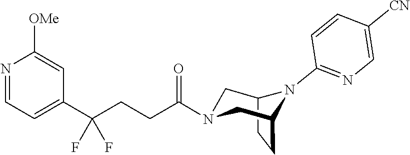

to obtain the crystallized compound.

In embodiments, water is added to the compound during the milling.

### Pharmaceutical Composition

In an aspect is provided a pharmaceutical composition including a crystalline compound of 6-(3-(4,4-difluoro-4-(2-methoxypyridin-4-yl)butanoyl)-3,8-diazabicyclo[3.2.1]octan-8-yl)nicotinonitrile having the structure

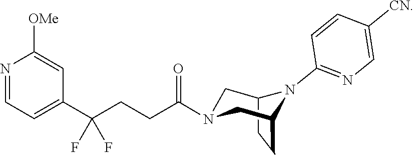

described herein and a pharmaceutically acceptable excipient.

In embodiments, the pharmaceutical composition includes an effective amount of the crystalline compound as described herein.

In an aspect, the pharmaceutical composition includes an active agent of the crystalline compound as described herein. The active agent is at least human muscarinic acetylcholine receptor M₁ (mAChR M₁) antagonist.

In embodiments, the crystalline compound is further processed to provide a more uniform particle size or to control the particle size or to reduce the particle size. For example, the initial crystalline material may be subject to mechanical impact means such as crushing, grinding, milling (such as ball milling and jet milling), and the like to provide particles having the desired particle size distribution.

In embodiments, the crystalline compound described herein is substantially pure, in that it contains less than about 5%, or less than about 1%, or less than about 0.1%, of other organic small molecules, such as contaminating intermediates or by-products that are created, for example, in one or more of the steps of a synthesis method. In embodiments, the crystalline compound described herein is substantially pure, in that it contains less than about 5% of other organic small molecules, such as contaminating intermediates or by-products that are created, for example, in one or more of the steps of a synthesis method. In embodiments, the crystalline compound described herein is substantially pure, in that it contains less than about 1% of other organic small molecules, such as contaminating intermediates or by-products that are created, for example, in one or more of the steps of a synthesis method. In embodiments, the crystalline compound described herein is substantially pure, in that it contains less than about 0.1% of other organic small molecules, such as contaminating intermediates or by-products that are created, for example, in one or more of the steps of a synthesis method.

These pharmaceutical compositions include those suitable for oral, rectal, topical, buccal, parenteral (e.g., subcutaneous, intramuscular, intradermal, or intravenous), vaginal, ophthalmic, or aerosol administration.

Exemplary pharmaceutical compositions are used in the form of a pharmaceutical preparation, for example, in solid, semisolid or liquid form, which includes the crystalline compound, as an active ingredient, in a mixture with an organic or inorganic carrier or excipient suitable for external, enteral or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active compound is included in the pharmaceutical composition in an amount sufficient to produce the desired effect upon the process or condition of the disease.

In some embodiments for preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid pre-formulation composition containing a homogeneous mixture of the crystalline compound described herein. When referring to these preformulation compositions as homogeneous, it is meant that the active ingredient is dispersed evenly throughout the composition so that the composition is readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, cellulose, microcrystalline cellulose, silicified microcrystalline cellulose, lactose, sucrose, glucose, mannitol, and/or silicic acid; (2) binders, such as, for example, carboxymethylcellulose, hypromellose, alginates, gelatin, polyvinyl pyrrolidone, sucrose and/or acacia; (3) humectants, such as glycerol; (4) disintegrating agents, such as crospovidone, croscarmellose sodium, sodium starch glycolate, agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, docusate sodium, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, in some embodiments, the compositions comprise buffering agents. In some embodiments, solid compositions of a similar type are also employed as fillers in soft and hard-filled gelatin capsules using such excipients as lactose or milk sugars, as well as high molecular weight polyethylene glycols and the like.

In some embodiments, a tablet is made by compression or molding, optionally with one or more accessory ingredients. In some embodiments, compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. In some embodiments, molded tablets are made by molding in a suitable machine a mixture of the subject composition moistened with an inert liquid diluent. In some embodiments, tablets, and other solid dosage forms, such as dragees, capsules, pills and granules, are scored or prepared with coatings and shells, such as enteric coatings and other coatings.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, in some embodiments, the liquid dosage forms contain inert diluents, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

In some embodiments, suspensions, in addition to the subject composition, contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, formulations for rectal or vaginal administration are presented as a suppository, which are prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. In some embodiments, the active component is mixed under sterile conditions with a pharmaceutically acceptable carrier, and with any preservatives, buffers, or propellants as required.

In some embodiments, the ointments, pastes, creams and gels contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In some embodiments, powders and sprays contain, in addition to a subject composition, excipients such as lactose, talc, silicic acid, aluminum hydroxide, calcium silicates and polyamide powder, or mixtures of these substances. In some embodiments, sprays additionally contain customary propellants, such as chlorofluorohydrocarbons and volatile unsubstituted hydrocarbons, such as butane and propane.

In some embodiments, the crystalline compound described herein is formulated as eye drops for ophthalmic administration.

Compositions and the crystalline compound disclosed herein alternatively are administered by aerosol. This is accomplished by preparing an aqueous aerosol, liposomal preparation or solid particles containing the crystalline compound. In some embodiments, a non-aqueous (e.g., fluorocarbon propellant) suspension is used. In some embodiments, sonic nebulizers are used because they minimize exposing the agent to shear, which results in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (e.g., Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which, in some embodiments, contain antioxidants, buffers, bacteriostats, solutes which render the formulation isotonic with the blood of the intended recipient or suspending or thickening agents.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants.

Also contemplated are enteral pharmaceutical formulations including the crystalline compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit 5100, Kollicoat EMM30D, Estacryl 30D, Coateric, and Aquateric). The solubility of each of the above materials is either known or is readily determinable in vitro.

The dose of the composition including the crystalline compound described herein differs, depending upon the patient's (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

Oral doses typically range from about 1.0 mg to about 1000 mg, one to four times, or more, per day.

The crystalline compound described herein is administered to subjects or patients (animals and humans) in need of such treatment in dosages that will provide optimal pharmaceutical efficacy. It will be appreciated that the dose required for use in any particular application will vary from patient to patient, not only with the particular composition selected, but also with the route of administration, the nature of the condition being treated, the age and condition of the patient, concurrent medication or special diets then being followed by the patient, and other factors, with the appropriate dosage ultimately being at the discretion of the attendant physician. For treating clinical conditions and diseases noted above, the crystalline compound disclosed herein is administered orally, subcutaneously, topically, parenterally, by inhalation spray or rectally in dosage unit formulations containing conventional non-toxic pharmaceutically acceptable carriers, adjuvants and vehicles.

Parenteral administration include subcutaneous injections, intravenous or intramuscular injections or infusion techniques.

### Method of Treatment

In an aspect is provided a method of treating, preventing, ameliorating, controlling or reducing the risk of a variety of disorders wherein the patient or subject would benefit from antagonism of the muscarinic acetylcholine M₁ receptor.

Antagonists of mAChR M₁

The muscarinic acetylcholine receptor M₁ (mAChR M₁) is found in both the central and peripheral nervous systems, particularly in the cerebral cortex and sympathetic ganglia. Notably, M₁ is expressed on OPCs in the central nervous system. Over time, OPCs will differentiate into myelin-producing oligodendrocytes. Myelin is indispensable for action potential conduction along the axon and its loss has been attributed to neuro-degenerative disorders, including multiple sclerosis. In embodiments, selective mAChR M₁ antagonists accelerate OPC differentiation into oligodendrocytes. In embodiments, selective mAChR M₁ antagonists are useful in the treatment of demyelinating disorders, such as multiple sclerosis. In embodiments, selective mAChR M₁ antagonists are useful in treating epileptic disorders and certain movement disorders, including Parkinson's disease, dystonia, and fragile X syndrome.

In one aspect, a treatment can include selective M₁ receptor antagonism to an extent effective to affect cholinergic activity. Thus, disorders for which the crystalline compound disclosed herein is useful can be associated with cholinergic activity, for example cholinergic hyperfunction. In embodiments, provided herein is a method of treating or preventing a disorder in a subject comprising the step of administering to the subject a crystalline compound of 6-(3-(4,4-difluoro-4-(2-methoxypyridin-4-yl)butanoyl)-3,8-diazabicyclo[3.2.1]octan-8-yl)nicotinonitrile described herein, or a pharmaceutical composition described herein in a dosage and amount effective to treat the disorder in the subject.

Provided herein is a method for the treatment of one or more disorders, for which muscarinic acetylcholine receptor inhibition is predicted to be beneficial, in a subject comprising the step of administering to the subject a crystalline compound of 6-(3-(4,4-difluoro-4-(2-methoxypyridin-4-yl)butanoyl)-3,8-diazabicyclo[3.2.1]octan-8-yl)nicotinonitrile described herein, or a pharmaceutical composition described herein in a dosage and amount effective to treat the disorder in the subject.

In embodiments provided herein is a method of treating a neurodegenerative disorder in a subject in need thereof, comprising administering to subject a therapeutically effective amount of a crystalline compound of 6-(3-(4,4-difluoro-4-(2-methoxypyridin-4-yl)butanoyl)-3,8-diazabicyclo[3.2.1]octan-8-yl)nicotinonitrile described herein.

In embodiments provided herein is a method of treating neuropathy in a subject in need thereof, comprising administering to the subject a therapeutically effective amount of a crystalline compound of 6-(3-(4,4-difluoro-4-(2-methoxypyridin-4-yl)butanoyl)-3,8-diazabicyclo[3.2.1]octan-8-yl)nicotinonitrile described herein. In embodiments is a method of treating neuropathy in a subject in need thereof, comprising administering to the subject a therapeutically effective amount of the crystalline compound described herein, wherein the neuropathy is peripheral neuropathy. In embodiments is a method of treating neuropathy in a subject in need thereof, comprising administering to the subject a therapeutically effective amount of the crystalline compound described herein, wherein the neuropathy is diabetic neuropathy.

In embodiments is a method of treating a demyelinating disease in a subject in need thereof, comprising administering to the subject a therapeutically effective amount of acrystalline compound of 6-(3-(4,4-difluoro-4-(2-methoxypyridin-4-yl)butanoyl)-3,8-diazabicyclo[3.2.1]octan-8-yl)nicotinonitrile described herein. In embodiments is a method of treating a demyelinating disease in a subject in need thereof, comprising administering to the subject a therapeutically effective amount of the crystalline compound described herein, wherein the demyelinating disease is a demyelinating disease of the central nervous system. In embodiments is a method of treating a demyelinating disease in a subject in need thereof, comprising administering to the subject a therapeutically effective amount of the crystalline compound described herein, wherein the demyelinating disease is multiple sclerosis. In embodiments is a method of treating a demyelinating disease in a subject in need thereof, comprising administering to the subject a therapeutically effective amount of the crystalline compound described herein, wherein the demyelinating disease is a demyelinating disease of the peripheral nervous system.

In embodiments is a method of modulating muscarinic acetylcholine receptor M₁ activity in a subject comprising administering to the subject a crystalline compound of 6-(3-(4,4-difluoro-4-(2-methoxypyridin-4-yl)butanoyl)-3,8-diazabicyclo[3.2.1]octan-8-yl)nicotinonitrile described herein. In embodiments, the crystalline compound described herein acts as a selective M₁ antagonist.

### Combination Therapy

Also contemplated herein are combination therapies, for example, co-administering the crystalline compound described herein and an additional active agent, as part of a specific treatment regimen intended to provide the beneficial effect from the co-action of these therapeutic agents. In embodiments, a crystalline compound of 6-(3-(4,4-difluoro-4-(2-methoxypyridin-4-yl)butanoyl)-3,8-diazabicyclo[3.2.1]octan-8-yl)nicotinonitrile described herein is administered in combination with one or more immunomodulatory agents. In embodiments, the crystalline compound described herein is administered in combination with one or more immunomodulatory agents, wherein the immunomodulatory agents are selected from an IFN-β 1 molecule; a corticosteroid; a polymer of glutamic acid, lysine, alanine and tyrosine or glatiramer; an antibody or fragment thereof against alpha-4 integrin or natalizumab; an anthracenedione molecule or mitoxantrone; a fingolimod or other S1P1 functional modulator; a dimethyl fumarate or other NRF2 functional modulator; an antibody to the alpha subunit of the IL-2 receptor of T cells (CD25) or daclizumab; an antibody against CD52 or alemtuzumab; an antibody against CD20 or ocrelizumab; and an inhibitor of a dihydroorotate dehydrogenase or teriflunomide. In embodiments, the immunomodulatory agent is an IFN-β 1 molecule. In embodiments, the immunomodulatory agent is a corticosteroid. In embodiments, the immunomodulatory agent is a polymer of glutamic acid, lysine, alanine and tyrosine or glatiramer. In embodiments, the immunomodulatory agent is an antibody or fragment thereof against alpha-4 integrin or natalizumab. In embodiments, the immunomodulatory agent is an anthracenedione molecule or mitoxantrone. In embodiments, the immunomodulatory agent is a fingolimod or other S1P1 functional modulator. In embodiments, the immunomodulatory agent is a dimethyl fumarate or other NRF2 functional modulator. In embodiments, the immunomodulatory agent is an antibody to the alpha subunit of the IL-2 receptor of T cells (CD25) or daclizumab. In embodiments, the immunomodulatory agent is an antibody against CD52 or alemtuzumab. In embodiments, the immunomodulatory agent is an antibody against CD20 or ocrelizumab. In embodiments, the immunomodulatory agent is an inhibitor of a dihydroorotate dehydrogenase or teriflunomide.

The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent) or in multiple, single formulations (e.g., capsules) for each of the therapeutic agents. Sequential or substantially simultaneous administration of each therapeutic agent is effected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

Combination therapy also embraces the administration of the therapeutic agents as described above in further combination with other biologically active ingredients and non-drug therapies. Where the combination therapy further comprises a non-drug treatment, the non-drug treatment is conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination are administered to a patient simultaneously or sequentially. It will be appreciated that the components are present in the same pharmaceutically acceptable carrier and, therefore, are administered simultaneously. Alternatively, the active ingredients are present in separate pharmaceutical carriers, such as conventional oral dosage forms, that are administered either simultaneously or sequentially.

The following examples are provided merely as illustrative of various embodiments and shall not be construed to limit the invention in any way.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Embodiments

Embodiment 1. A crystalline compound having the formula:

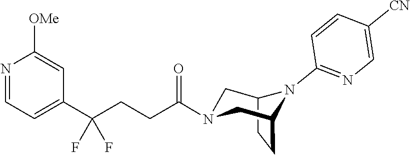

characterized by an x-ray powder diffraction pattern with peaks at 13.4±0.15° 2θ, 14.0±0.15° 2θ, 15.2±0.15° 2θ, 19.3±0.15° 2θ, 20.0±0.15° 2θ and 21.2±0.150 20.

Embodiment 2. The crystalline compound of Embodiment 1, wherein the compound is characterized by an x-ray powder diffraction pattern with peaks at 6.7±0.15° 2θ, at 13.4±0.15° 2θ, 14.0±0.15° 2θ, 15.2±0.15° 2θ, 17.7±0.15° 2θ, 18.1±0.15° 2θ, 18.8±0.15° 2θ, 19.3±0.15° 2θ, 19.8±0.15° 2θ, 20.0±0.15° 2θ, 21.0±0.15° 2θ, 21.2±0.15° 2θ, and 24.1±0.15° 2θ.

Embodiment 3. The crystalline compound of Embodiment 1, wherein the compound is characterized by an x-ray powder diffraction pattern with peaks at 6.7±0.15° 2θ, 10.7±0.15° 2θ, 13.4±0.15° 2θ, 14.0±0.15° 2θ, 15.2±0.15° 2θ, 17.7±0.15° 2θ, 18.1±0.15° 2θ, 18.8±0.15° 2θ, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 27.8±0.15° 2θ, and 30.6±0.15° 2θ.

Embodiment 4. The crystalline compound of Embodiment 1, wherein the compound is characterized by an x-ray powder diffraction pattern with peaks at 6.7±0.150 20, 10.7±0.15° 2θ, 13.4±0.15° 2θ, 14.0±0.15° 2θ, 15.2±0.15° 2θ, 16.6±0.15° 2θ, 17.7±0.15° 2θ, 18.1±0.15° 2θ, 18.4±0.15° 2θ, 18.8±0.15° 2θ, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 26.3±0.15° 2θ, 26.6±0.15° 2θ, 27.0±0.15° 2θ, 27.8±0.15° 2θ, and 30.6±0.15° 20.

Embodiment 5. The crystalline compound of Embodiment 1, wherein the compound is further characterized as having a differential scanning calorimetry endotherm onset at about 117° C.

Embodiment 6. A pharmaceutical composition comprising a crystalline compound of any one of Embodiments 1 to 5 and at least one pharmaceutically acceptable excipient.

Embodiment 7. A method of treating a neurodegenerative disorder in a subject in need thereof, comprising administering to the subject a therapeutically effective amount of a crystalline compound of any one of Embodiments 1 to 5.

Embodiment 8. The method of Embodiment 7, further comprising the administration of one or more immunomodulatory agents.

Embodiment 9. The method of Embodiment 8, wherein the one or more immunomodulatory agents are selected from: an IFN-β 1 molecule; a corticosteroid; a polymer of glutamic acid, lysine, alanine and tyrosine or glatiramer; an antibody or fragment thereof against alpha-4 integrin or natalizumab; an anthracenedione molecule or mitoxantrone; a fingolimod or FTY720 or other S1P1 functional modulator; a dimethyl fumarate or other NRF2 functional modulator; an antibody to the alpha subunit of the IL-2 receptor of T cells (CD25) or daclizumab; an antibody against CD52 or alemtuzumab; an antibody against CD20 or ocrelizumab; and an inhibitor of a dihydroorotate dehydrogenase or teriflunomide.

Embodiment 10. A method of treating a demyelinating disease in a subject in need thereof, comprising administering to the subject a therapeutically effective amount of a crystalline compound of any one of Embodiments 1 to 5.

Embodiment 11. The method of Embodiment 10, wherein the demyelinating disease is a demyelinating disease of the central nervous system.

Embodiment 12. The method of Embodiment 11, wherein the demyelinating disease is multiple sclerosis.

Embodiment 13. The method of Embodiment 10, wherein the demyelinating disease is a demyelinating disease of the peripheral nervous system.

Embodiment 14. The method of Embodiment 10, further comprising the administration of one or more immunomodulatory agents.

Embodiment 15. The method of Embodiment 14, wherein the one or more immunomodulatory agents are selected from: an IFN-β 1 molecule; a corticosteroid; a polymer of glutamic acid, lysine, alanine and tyrosine or glatiramer; an antibody or fragment thereof against alpha-4 integrin or natalizumab; an anthracenedione molecule or mitoxantrone; a fingolimod or FTY720 or other S1P1 functional modulator; a dimethyl fumarate or other NRF2 functional modulator; an antibody to the alpha subunit of the IL-2 receptor of T cells (CD25) or daclizumab; an antibody against CD52 or alemtuzumab; an antibody against CD20 or ocrelizumab; and an inhibitor of a dihydroorotate dehydrogenase or teriflunomide.

Embodiment 16. A method of treating a neuropathic disease, optionally a peripheral neuropathy, in a subject in need thereof, comprising administering to the subject a therapeutically effective amount of a crystalline compound of any one of Embodiments 1 to 5.

Embodiment 17. The method of Embodiment 16, wherein the neuropathic disease is diabetic neuropathy.

Embodiment 18. The method of Embodiment 16, further comprising the administration of one or more immunomodulatory agents.

Embodiment 19. The method of Embodiment 18, wherein the one or more immunomodulatory agents are selected from: an IFN-β 1 molecule; a corticosteroid; a polymer of glutamic acid, lysine, alanine and tyrosine or glatiramer; an antibody or fragment thereof against alpha-4 integrin or natalizumab; an anthracenedione molecule or mitoxantrone; a fingolimod or FTY720 or other S1P1 functional modulator; a dimethyl fumarate or other NRF2 functional modulator; an antibody to the alpha subunit of the IL-2 receptor of T cells (CD25) or daclizumab; an antibody against CD52 or alemtuzumab; an antibody against CD20 or ocrelizumab; and an inhibitor of a dihydroorotate dehydrogenase or teriflunomide.

Embodiment 20. A method of modulating muscarinic acetylcholine receptor M₁ activity in a subject comprising administering to the subject a crystalline compound of any one of Embodiments 1 to 5.

Embodiment 21. The method of Embodiment 20, wherein the compound acts as a selective M₁ antagonist.

### EXAMPLES

### Characterization of Crystalline Compound

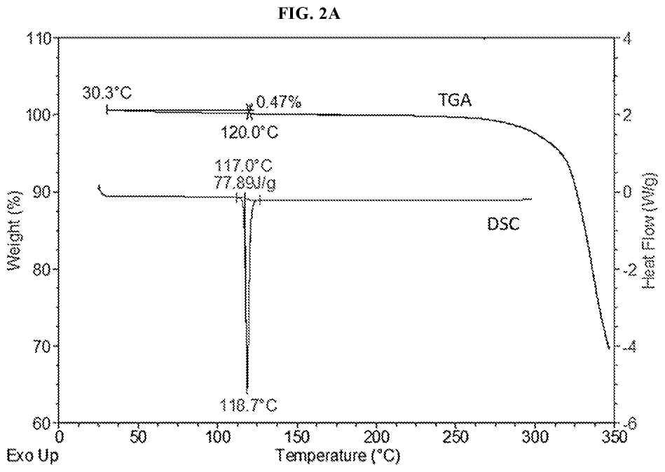

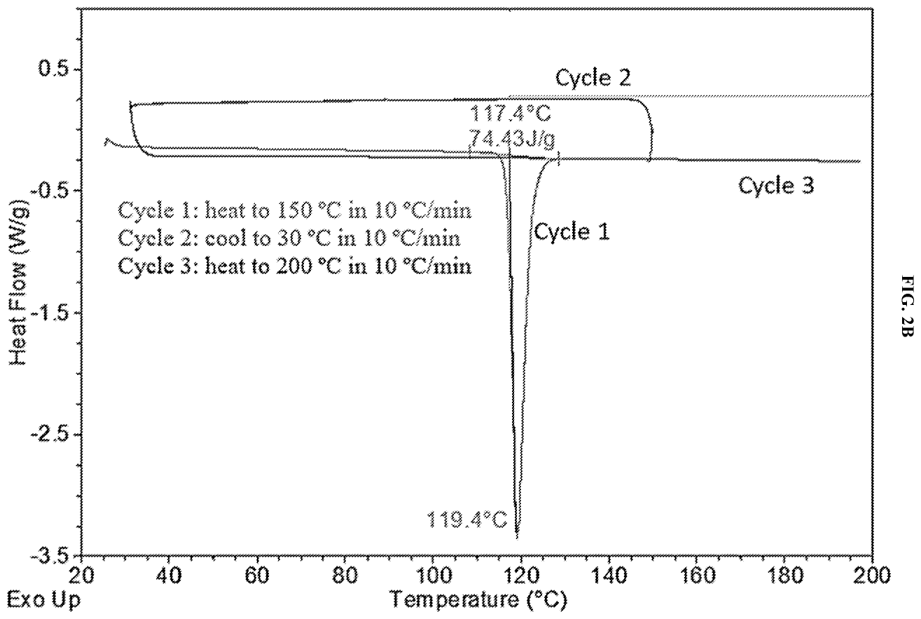

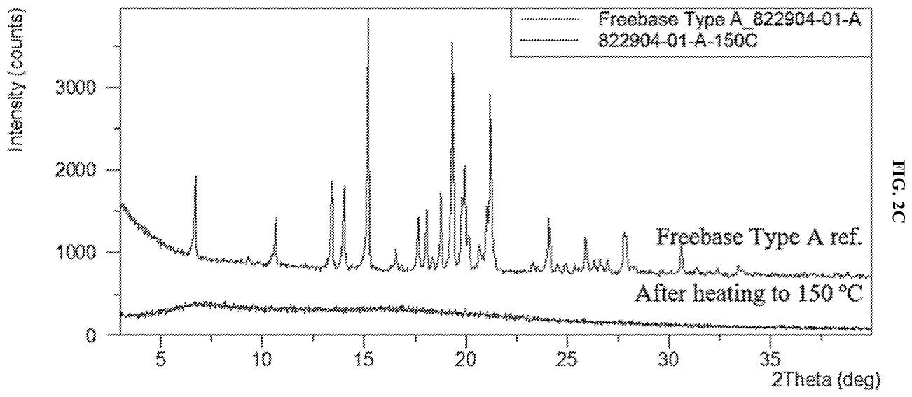

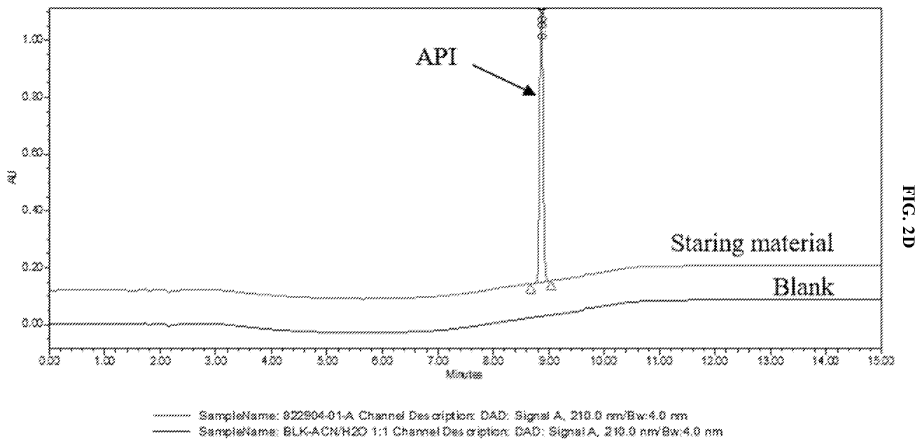

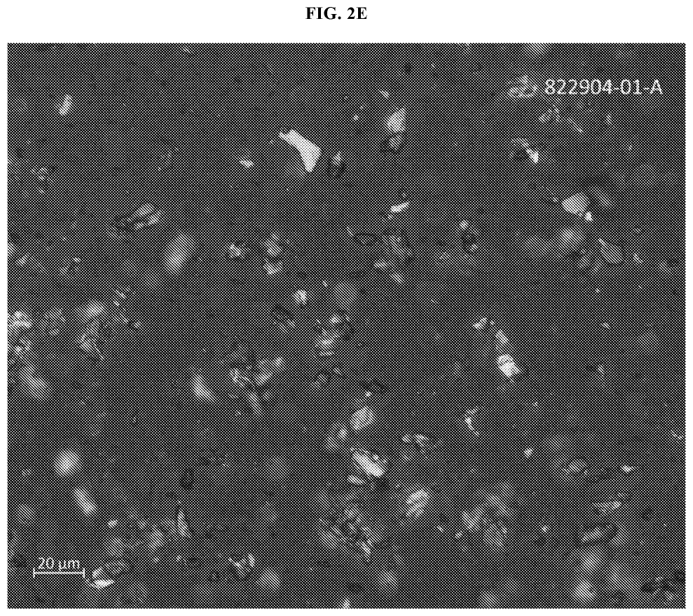

The crystalline compound described herein was characterized by X-ray powder diffraction (XRPD), thermo gravimetric analysis (TGA), differential scanning calorimetry (DSC), cycle-DSC, polarized light microscopy (PLM), and high performance liquid chromatography (HPLC). The crystalline compound was found to exhibit a XRPD pattern shown in FIG. 1, with the peaks listed in Table 1. This crystalline form was hereby designated as “Freebase Type A”. TGA/DSC results in FIG. 2A showed a weight loss of 0.5% up to 120° C., and one endothermic signal at 117.0° C. (onset). Thus, with limited TGA loss before 120° C. and a neat DSC signal, Freebase Type A was speculated to be an anhydrate. Cycle-DSC curve in FIG. 2B showed that only one endothermic signal at 117.4° C. (onset) was observed in cycle 1 (heat to 150° C. in 10° C./min) and no thermal signal was detected in cycle 2 (cool to 30° C. in 10° C./min) nor cycle 3 (heat to 200° C. in 10° C./min). XRPD result in FIG. 2C showed that after heating to 150° C. and cooling to RT, amorphous sample was obtained. Combined with the cycle-DSC data, it was speculated that it might be hard for the crystalline compound to re-crystalize spontaneously after melting. HPLC chromatogram in FIG. 2D showed that the HPLC purity of the crystalline compound was 100.00 area %. PLM image in FIG. 2E showed that the morphology of the crystalline compound was plate-like.

Further evaluations, including kinetic solubility at 37° C., physicochemical stability, and hygroscopicity evaluation were performed on Freebase Type A.

### 1. Kinetic Solubility Evaluation

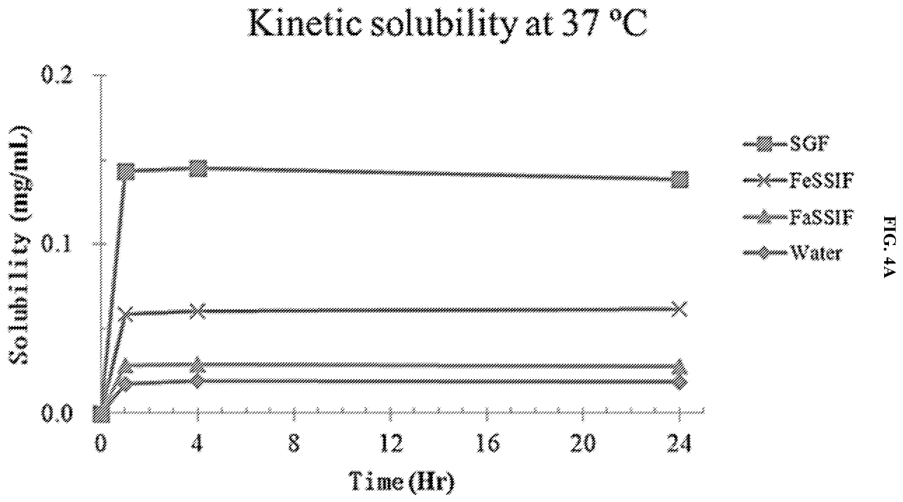

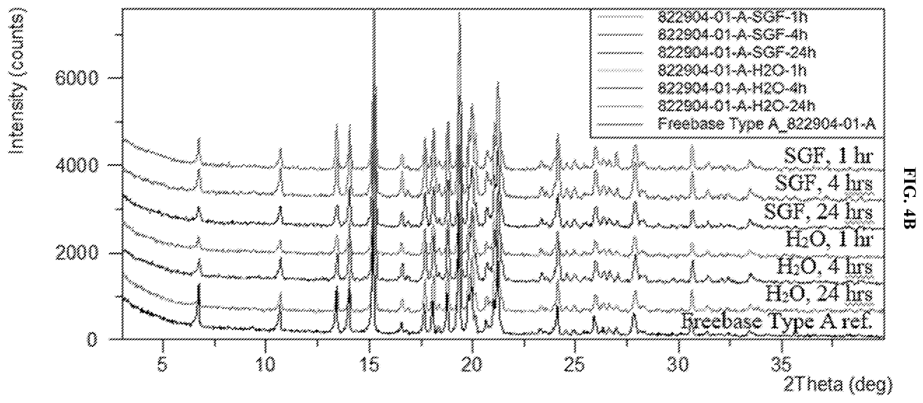

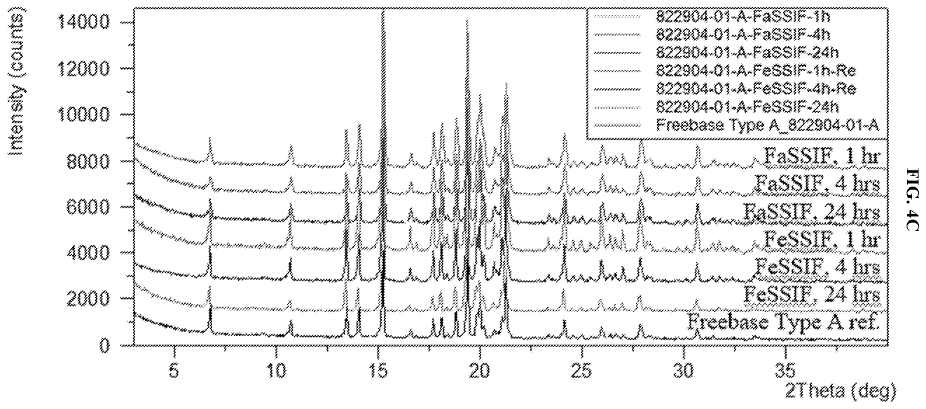

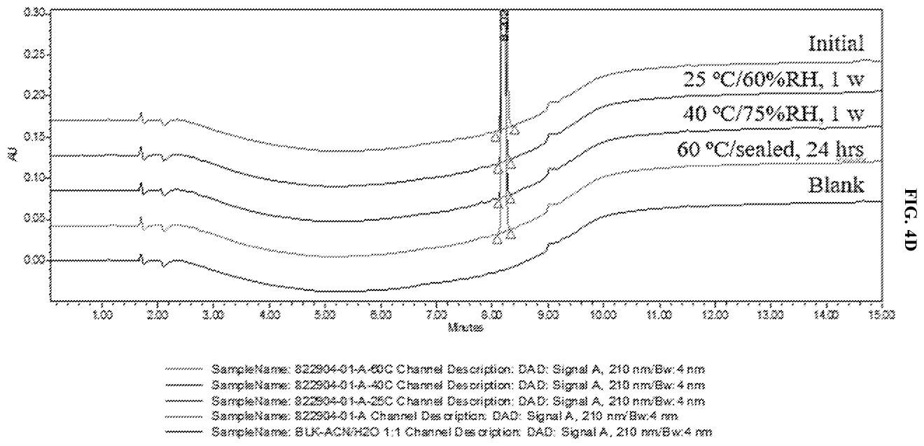

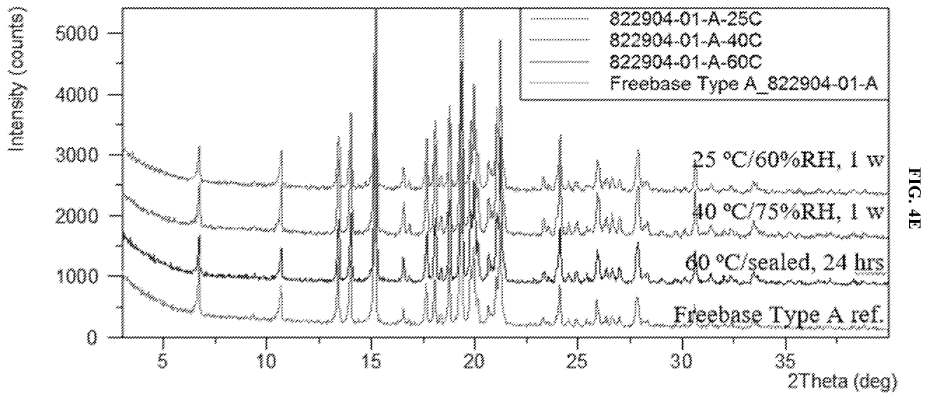

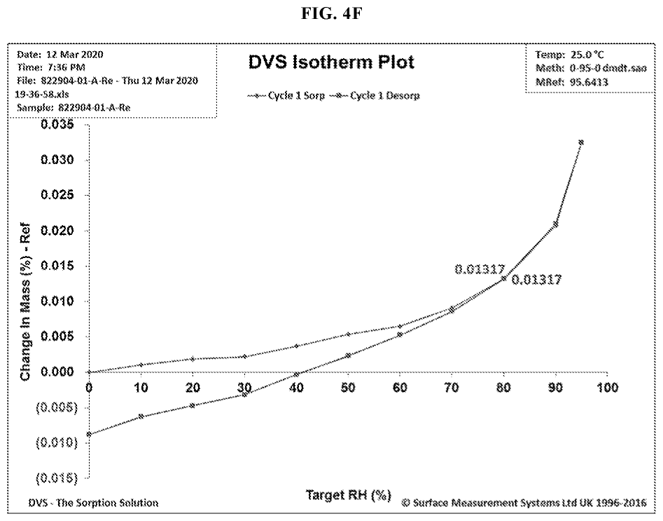

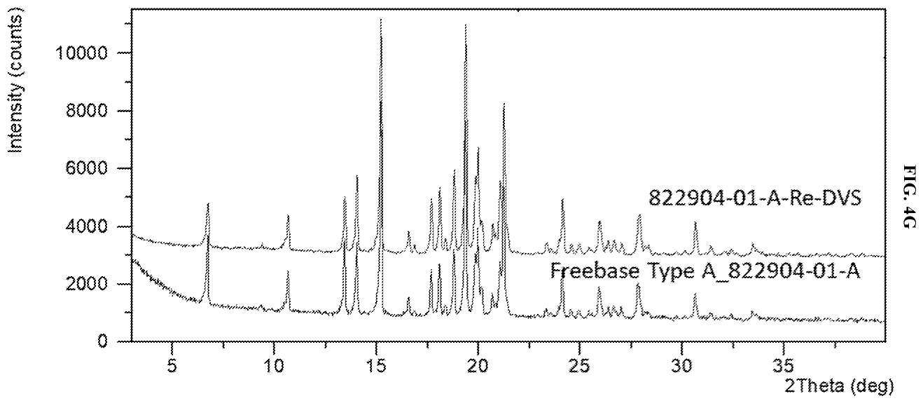

In these experiments, about 20 mg of Freebase Type A was suspended in 4 mL of FaSSIF, FeSSIF, SGF and H₂O, respectively. After rolling at 37° C. for 1 hr, 4 hrs and 24 hrs, the suspension was centrifuged and filtered through a 0.45 μm PTFE membrane. The residual solids were tested by XRPD and the supernatant was tested by HPLC and pH meter. Detailed procedure can be found in Table 15. Results showed that the solubility of freebase Type A was ranked in the order of SGF>FeSSIF>FaSSIF>H₂O, from high to low. The solubility in SGF was up to 0.15 mg/mL and the solubility in H₂O was 0.02 mg/mL. XRPD results in FIG. 4B and FIG. 4C showed that after rolling in tested media for 1, 4, and 24 hrs, no form change was observed. The results of kinetic solubility evaluation are summarized in FIG. 4A and Table 2.

TABLE 2
Summary of kinetic solubility evaluation

| | | 1 hr | | | 4 hrs | | | 24 hrs | | |
|---|---|---|---|---|---|---|---|---|---|---|
| Material | Media | S | | | S | | | S | | |
| (ID: 822904-) | (pH) | (mg/mL) | pH | FC | (mg/mL) | pH | FC | (mg/mL) | pH | FC |
| Freebase Type | SGF | 0.14 | 1.85 | No | 0.15 | 1.77 | No | 0.14 | 1.82 | No |
| A (01-A) | (1.77) | | | | | | | | | |
| | H₂O | 0.02 | 7.16 | No | 0.02 | 6.65 | No | 0.02 | 7.19 | No |
| | (6.95) | | | | | | | | | |
| | FaSSIF | 0.03 | 6.53 | No | 0.03 | 6.45 | No | 0.03 | 6.48 | No |
| | (6.47) | | | | | | | | | |
| | FeSSIF | 0.06 | 5.01 | No | 0.06 | 4.97 | No | 0.06 | 5.01 | No |
| | (4.99) | | | | | | | | | |

S: Solubility of freebase;
FC: Form change.

### 2. Physicochemical Stability Evaluation

In these experiments, about 10 mg of Freebase Type A was stored at 25° C./60% RH, 40° C./75% RH for one week and 60° C./sealed for 24 hrs. After storage, the solids were tested by XRPD and HPLC. As shown by the HPLC results in FIG. 4D, no purity decrease was detected in Freebase Type A. XPRD overlay in FIG. 4E showed that no form change was observed. The results are summarized in Table 3.

TABLE 3
Summary of physicochemical stability evaluation

| | | | HPLC purity | | |
|---|---|---|---|---|---|
| Material | | Initial | after storage | Of | Form |
| (ID 822904-) | Condition | (area %) | (area %) | initial | change |
| Freebase Type A | 25° C./60% RH, 1 w | 100.00 | 100.00 | 100.00% | No |
| (01-A) | 40° C./75% RH, 1 w | | 100.00 | 100.00% | No |
| | 60° C./sealed, 24 hrs | | 100.00 | 100.00% | No |

### 3. Hygroscopicity Evaluation

Hygroscopicity of Freebase Type A was evaluated by DVS test. As shown in FIG. 4F, 0.01% water uptake of Freebase Type A was detected at 80% RH/25° C. in the sorption cycle from 0% RH to 95% RH, which indicated that “Freebase Type A was non-hygroscopic”. For the minus weight change observed in the desorption curve, it might be related to sample loss or instrument fluctuation. XRPD overlay in FIG. 4G showed that no form change was observed after the DVS test.

### Polymorph Screening Experiments

A total of 102 polymorph screening experiments were conducted via various crystallization methods. Results in Table 5 showed that only Freebase Type A was obtained. The solvent abbreviations used in the screening are listed in Table 4.

TABLE 4
Solvent abbreviation list

| Abbrevia- | | Abbrevia- | |
|---|---|---|---|
| tion | Solvent | tion | Solvent |
| MeOH | Methanol | MTBE | Methyl tert-butyl ether |
| EtOH | Ethanol | THF | Tetrahydrofuran |
| IPA | Isopropyl alcohol | DMSO | Dimethylsulfoxide |
| MEK | Methyl ethyl ketone | DMF | N,N-Dimethylformamide |
| MIBK | Methyl isobutyl ketone | ACN | Acetonitrile |
| EtOAc | Ethyl acetate | DCM | Dichloromethane |
| IPAc | Isopropyl acetate | 2-MeTHF | 2-Methyltetrahydrofuran |
| CHCl₃ | Chloroform | CPME | Cyclopentyl methyl ether |
| n-BuOH | n-Butyl alcohol | — | — |

TABLE 5
Summary of polymorph screening experiments

| | No. of | |
|---|---|---|
| Method | Experiment | Results |
| Anti-solvent addition | 12 | Freebase Type A, freebase Type |
| | | A + extra peak* |
| Evaporation | 12 | Freebase Type A |
| Temperature cycling | 13 | Freebase Type A, gel |
| Slurry at RT | 24 | Freebase Type A, gel |
| Slurry at 50° C. | 10 | Freebase Type A |
| Solid vapor diffusion | 12 | Freebase Type A |
| Liquid vapor diffusion | 9 | Freebase Type A, gel |
| Polymer-induced | 8 | Freebase Type A, amorphous |
| Crystallization | | |
| Grinding | 2 | Freebase Type A |
| Total | 102 | Freebase Type A, freebase Type |
| | | A + extra peak*, amorphous, gel |

*After storage at RT for ~1 week, the extra peak disappeared.

### 1. Anti-Solvent Addition

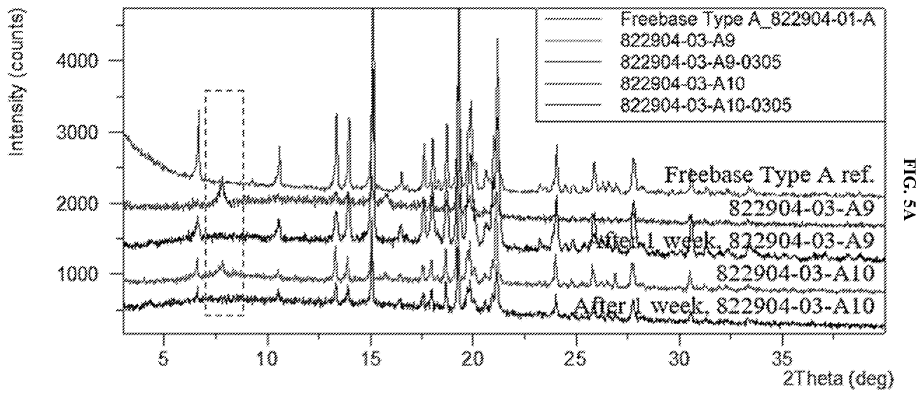

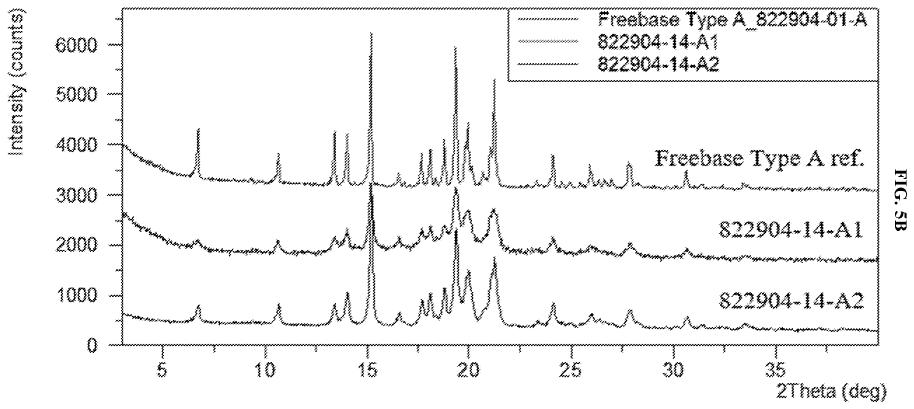

A total of twelve anti-solvent addition experiments were carried out. About 20 mg of Freebase Type A was dissolved in a 20-mL vial with the corresponding solvents listed in Table 6. After filtration through a 0.45 μm PTFE membrane, the obtained clear solution was magnetically stirred at a rate of 750 rpm at RT and then added the corresponding anti-solvent listed in Table 6 to induce precipitation, or until the total amount of anti-solvent reached 4.8 mL. The sample was stirred at RT for 1˜2 hrs before the solid was isolated and analyzed. Results are summarized in Table 6. For the samples with an extra peak (as marked), as shown in FIG. 5A, after storage at RT for ˜1 week, the extra peak disappeared.

TABLE 6
Summary of anti-solvent addition experiments

| Experiment ID | Solvent | Anti-solvent | Results |
|---|---|---|---|
| 822904-03-A1 | ACN | H₂O | Freebase Type A |
| 822904-03-A2 | THF | | Freebase Type A |
| 822904-03-A3 | DMSO | | Freebase Type A |
| 822904-03-A4 | MeOH | | Freebase Type A |
| 822904-03-A5 | IPAc | n-Hexane | Freebase Type A |
| 822904-03-A6 | Anisole | | Freebase Type A |
| 822904-03-A7 | MIBK | | Freebase Type A |
| 822904-03-A8 | DCM | | Freebase Type A |
| 822904-03-A9 | Toluene | n-Heptane | Freebase Type A + extra peak |
| 822904-03-A10 | Acetone | | Freebase Type A + extra peak |
| 822904-03-A11 | EtOAc | | Freebase Type A |
| 822904-03-A12 | CHCl₃ | Cyclohexane | Freebase Type A |

### 2. Slow Evaporation

Slow evaporation experiments were performed under twelve conditions with different solvent systems. Briefly, about 20 mg of Freebase Type A was dissolved in a 3-mL vial with the corresponding solvents listed in Table 7. After vortex and filtration through a 0.45 μm PTFE membrane, the obtained clear solution was sealed with Parafilm® pierced with a tiny hole and allowed to slowly evaporate at RT. Resulting solids were isolate for XRPD analysis. Results are summarized in Table 7.

TABLE 7
Summary of slow evaporation experiments

| Experiment ID | Solvent, v:v | Solid Form |
|---|---|---|
| 822904-04-A1 | MeOH | Freebase Type A |
| 822904-04-A2 | DCM | Freebase Type A |
| 822904-04-A3 | Acetone | Freebase Type A |
| 822904-04-A4 | ACN | Freebase Type A |
| 822904-04-A5 | THF | Freebase Type A |
| 822904-04-A6 | MTBE | Freebase Type A |
| 822904-04-A7 | EtOAc | Freebase Type A |
| 822904-04-A8 | MEK | Freebase Type A |
| 822904-04-A9 | H₂O/MeOH, 1:1 | Freebase Type A |
| 822904-04-A10 | Cyclohexane/EtOAc, 2:1 | Freebase Type A |
| 822904-04-A11 | n-BuOH/ACN, 2:1 | Freebase Type A |
| 822904-04-A12 | IPA/2-MeTHF, 2:1 | Freebase Type A |

### 3. Temperature Cycling (50° C. to 5° C.)

Temperature cycling experiments were conducted in thirteen solvent systems. About 20 mg of Freebase Type A was suspended in an HPLC vial with 0.5 mL of the corresponding solvents listed in Table 8. Cycling procedure: ramp to 50° C. at a rate of 4.5° C./min, keep the temperature at 50° C. for 30 min; cool down to 5° C. at a rate of 0.1° C./min and keep the temperature at 5° C. for 30 min. After three cycles, the samples were stored at 5° C. The obtained solids were isolated for XRPD analysis. If a clear solution was obtained, the sample was slurried at −20° C. If there were still no visible precipitates, the solution was allowed to evaporate slowly at RT. Results are summarized in Table 8.

TABLE 8
Summary of temperature cycling experiments

| Experiment ID | Solvent, v:v | Solid Form |
|---|---|---|
| 822904-05-A1 | EtOH | Freebase Type A |
| 822904-05-A2 | IPA | Freebase Type A |
| 822904-05-A3 | MTBE | Freebase Type A |
| 822904-05-A4 | CPME | Freebase Type A |
| 822904-05-A5 | n-Propanol | Freebase Type A |
| 822904-05-A6 | H₂O | Freebase Type A |
| 822904-05-A7 | MEK/n-BuOH, 1:1 | Freebase Type A* |
| 822904-05-A8 | EtOAc/Cyclohexane, 1:1 | Freebase Type A |
| 822904-05-A9 | Acetone/n-Hexane, 1:1 | Freebase Type A* |
| 822904-05-A10 | MIBK/n-Heptane, 3:1 | Freebase Type A |
| 822904-05-A11 | n-Heptane/Anisole, 3:1 | Freebase Type A |
| 822904-05-A12 | IPAc/n-Heptane, 3:1 | Freebase Type A* |
| 822904-05-A13 | ACN/H₂O, 3:1 | Gel* |

*Transfer to slurry at −20° C. → slow evaporation at RT (3 holes).

### 4. Slurry Conversion at RT

Slurry conversion experiments were conducted at RT in twenty-four different solvent systems. About 20 mg of Freebase Type A was suspended in an HPLC vial with 0.5 mL of the corresponding solvents listed in Table 9. The suspension was magnetically stirred at a speed of ˜750 rpm at RT. After ˜1 week, the remaining solids were isolated for XRPD analysis. If only a few solids or a clear solution was obtained, the sample was slurried at 5° C. If the sample was still a clear solution, it was slurried at −20° C. If there were still no visible precipitates, the solution was allowed to evaporate slowly at RT. Results are summarized in Table 9.

TABLE 9
Summary of slurry conversion experiments at RT

| Experiment ID | Solvent, v:v | Solid Form |
|---|---|---|
| 822904-06-A1 | EtOH | Freebase Type A |
| 822904-06-A2 | IPA | Freebase Type A |
| 822904-06-A3 | MTBE | Freebase Type A |
| 822904-06-A4 | CPME | Freebase Type A |
| 822904-06-A5 | Dimethoxyethane/CPME, 1:1 | Gel *** |
| 822904-06-A6 | MIBK/EtOH, 2:1 | Freebase Type A*** |
| 822904-06-A7 | IPAc/MTBE, 2:1 | Freebase Type A** |
| 822904-06-A8 | ACN/MTBE, 1:2 | Freebase Type A*** |
| 822904-06-A9 | m-Xylene/n-Heptane, 2:1 | Freebase Type A |
| 822904-06-A10 | Methyl acetate/EtOH, 1:4 | Freebase Type A* |
| 822904-06-A11 | EtOAc/n-Heptane, 1:9 | Freebase Type A |
| 822904-06-A12 | DMSO/H₂O, 1:9 | Freebase Type A |
| 822904-06-A13 | MEK/n-Heptane, 1:4 | Freebase Type A |
| 822904-06-A14 | CHCI3/n-Hexane, 1:9 | Freebase Type A |
| 822904-06-A15 | 1,4-Dioxane/n-Hexane, 1:4 | Freebase Type A |
| 822904-06-A16 | Toluene/n-Hexane, 1:4 | Freebase Type A |
| 822904-06-A17 | 2-MeTHF/Cyclohexane, 1:4 | Freebase Type A |
| 822904-06-A18 | DCM/Cyclohexane, 1:9 | Freebase Type A |
| 822904-06-A19 | Acetone | Freebase Type A*** |
| 822904-06-A20 | Acetone/H₂O, 98:2 | Freebase Type A*** |
| 822904-06-A21 | Acetone/H₂O, 95:5 | Freebase Type A*** |
| 822904-06-A22 | Acetone/H₂O, 86:14 | Freebase Type A*** |
| 822904-06-A23 | Acetone/H₂O, 6:4 | Freebase Type A |
| 822904-06-A24 | H₂O | Freebase Type A |

*Transfer to slurry at 5° C.;
**Transfer to slurry at 5° C. → slurry at −20° C.;
***Transfer to slurry at 5° C. → slurry at −20° C. → slow evaporation at RT (3 holes).

### 5. Slurry Conversion at 50° C.

Slurry conversion experiments were conducted at 50° C. in ten solvent systems. About 20 mg of Freebase Type A was suspended in an HPLC glass vial with 0.5 mL of the corresponding solvents listed in Table 10. The suspension was kept stirring at 50° C. If there were no visible solids after the slurry, the sample was stirred further at RT. If the sample was still clear, it was slurried at −20° C. For samples that still did not afford visible precipitates, they were allowed to evaporate slowly at RT. Resulting solids were isolated for XRPD analysis. Results are summarized in Table 10.

TABLE 10
Summary of slurry conversion experiments at 50° C.

| Experiment ID | Solvent, v:v | Solid Form |
|---|---|---|
| 822904-07-A1 | IPA | Freebase Type A |
| 822904-07-A2 | EtOH | Freebase Type A* |
| 822904-07-A3 | H₂O | Freebase Type A |
| 822904-07-A4 | MTBE | Freebase Type A |
| 822904-07-A5 | Methyl acetate/n-Hexane, | Freebase Type A** |
| | 2:1 | |
| 822904-07-A6 | CHCl₃/n-Heptane, 2:1 | Freebase Type A*** |
| 822904-07-A7 | MEK/n-Hexane, 2:1 | Freebase Type A** |
| 822904-07-A8 | ACN/n-Hexane, 2:1 | Freebase Type A*** |
| 822904-07-A9 | Cyclohexane | Freebase Type A |
| 822904-07-A10 | n-Heptane | Freebase Type A |

*Transfer to slurry at RT;
**Transfer to slurry at RT → slurry at −20° C.;
***Transfer to slurry at RT → slurry at −20° C. → slow evaporation at RT (3 holes).

### 6. Solid Vapor Diffusion

A total of twelve solid vapor diffusion experiments were carried out in different solvents. About 20 mg of Freebase Type A was weighed into a 4-mL glass vial. This vial was then carefully placed unsealed in a 20-mL glass vial containing 4 mL of the corresponding solvents listed in Table 11. The 20-mL vial was sealed and kept at RT for 2-3 weeks to allow for sufficient interaction between the solvent vapor and the solid. The resulting solids were tested by XRPD. If a clear solution was obtained instead, the sample was allowed to slowly evaporate at RT. Results are summarized in Table 11.

TABLE 11
Summary of solid vapor diffusion experiments

| Experiment ID | Solvent | Solid Form |
|---|---|---|
| 822904-08-A1 | H₂O | Freebase Type A |
| 822904-08-A2 | DCM | Freebase Type A* |
| 822904-08-A3 | CHCl₃ | Freebase Type A* |
| 822904-08-A4 | MeOH | Freebase Type A |
| 822904-08-A5 | EtOH | Freebase Type A |
| 822904-08-A6 | ACN | Freebase Type A* |
| 822904-08-A7 | Acetone | Freebase Type A* |
| 822904-08-A8 | EtOAc | Freebase Type A* |
| 822904-08-A9 | MTBE | Freebase Type A |
| 822904-08-A10 | MEK | Freebase Type A* |
| 822904-08-A11 | THF | Freebase Type A* |
| 822904-08-A12 | DMSO | Freebase Type A |

*Transfer to slow evaporation at RT (3 holes).

### 7. Liquid Vapor Diffusion

Liquid vapor diffusion experiments were performed under nine conditions. About 20 mg of Freebase Type A was dissolved in the corresponding solvents listed in Table 12. After filtration, the clear solution was transferred to a 4-mL glass vial. This vial was then carefully placed unsealed in a 20-mL glass vial containing 4 mL of the corresponding anti-solvents listed in Table 12. The 20-mL vial was sealed and kept at RT to allow for sufficient interaction between solvent vapor and the sample solution. The resulting solids were isolated for XRPD analysis and results are summarized in Table 12.

TABLE 12
Summary of solution vapor diffusion experiments

| Experiment ID | Solvent | Anti-solvent | Results |
|---|---|---|---|
| 822904-09-A1 | 2-MeTHF | n-Hexane | Freebase Type A |
| 822904-09-A2 | MEK | | Freebase Type A |
| 822904-09-A3 | EtOAc | | Freebase Type A |
| 822904-09-A4 | 1,4-Dioxane | H₂O | Gel |
| 822904-09-A5 | Toluene | Cyclohexane | Freebase Type A |
| 822904-09-A6 | IPAC | | Freebase Type A |
| 822904-09-A7 | Anisole | | Freebase Type A |
| 822904-09-A8 | EtOH | n-Pentane | Freebase Type A |
| 822904-09-A9 | CHCl₃ | | Freebase Type A |

### 8. Polymer-Induced Crystallization Experiments

Polymer induced crystallization experiments were conducted at RT in eight solvent systems. About 20 mg of Freebase Type A was suspended with 0.5 mL of the corresponding solvents listed in Table 18 in a 3-mL vial. The samples were slurried at RT. After filtration, the clear solution was transferred to a 3-mL glass vial containing 2 mg of the corresponding polymer mixture listed in Table 13. The vial was then sealed with Parafilm® pierced with a tiny hole to facilitate slow evaporation at RT. The solid was then isolated for XRPD analysis. Results are summarized in Table 13.

TABLE 13
Summary of polymer-induced crystallization experiments

| | | Polymer | |
|---|---|---|---|
| Experiment ID | Solvent, v:v | Mixture | Results |
| 822904-10-A1 | IPAc | A | Freebase Type A |
| 822904-10-A2 | H₂O/ACN, 1:1 | | Amorphous |
| 822904-10-A3 | EtOH/CHCl₃, 2:1 | | Freebase Type A |
| 822904-10-A4 | n-Hexane/Acetone, | | Freebase Type A |
| | 2:1 | | |
| 822904-10-A5 | CHCl₃ | | Freebase Type A |
| 822904-10-A6 | MEK | B | Freebase Type A |
| 822904-10-A7 | MTBE/DCM, 2:1 | | Freebase Type A |
| 822904-10-A8 | 1,4-Dioxane/H₂O, | | Freebase Type A |
| | 2:1 | | |

Polymer mixture A: Polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1)
Polymer mixture B: Polycaprolactone (PCL), polyethylene glycol (PEG), poly (methyl methacrylate) (PMMA), sodium alginate (SA), hydroxyethyl cellulose (HEC) (mass ratio of 1:1:1:1:1)

### 9. Grinding Experiments

Grinding experiments were conducted under two conditions (dry and with H₂O). Dry: about 20 mg of Freebase Type A was manually milled for ˜15 min and the resulting solid was tested by XRPD. Wet: about 20 mg of Freebase Type A was manually milled with 2 drops of H₂O for ˜15 min and then the resulting solid was tested by XRPD. Results are summarized in Table 14 and the XRPD overlay is displayed in FIG. 5B.

TABLE 14
Summary of grinding experiments

| Experiment ID | Solvent | Solid Form |
|---|---|---|
| 822904-14-A1 | NA | Freebase Type A |
| 822904-14-A2 | H₂O | Freebase Type A |

### Procedures for Kinetic Solubility Evaluation

The detailed procedures for kinetic solubility evaluation are summarized in Table 15.

TABLE 15
Detailed procedures for kinetic solubility evaluation

| Items | Conditions |
|---|---|
| Media | SGF: Weigh 201.3 mg of sodium chloride (NaCl) and 103.2 |
| Prepara- | mg of Triton X-100 into a 100-mL volumetric flask. Add |
| tion | appropriate volume of purified water and sonicate until all |
| | solids are completely dissolved. Add about 1.632 mL of 1M |
| | HCl and sufficient purified water closely to the target volume |
| | and adjust to pH 1.81. Dilute to volume with purified water, |
| | mix well. |
| | FaSSIF: FaSSIF dissolving buffer: weigh 339.7 mg of |
| | sodium phosphate monobasic, 43.0 mg of sodium hydroxide |
| | and 620.7 mg of sodium chloride into a 100-mL volumetric |
| | flask. Add appropriate volume of purified water and sonicate |
| | until all solids are completely dissolved. Add sufficient |
| | purified water closely to the target volume and adjust to pH |
| | 6.49. Dilute to volume with purified water, mix well. |
| | Weigh 220.0 mg of SIF powder into a 100-mL volumetric |
| | flask. Add appropriate volume of FaSSIF dissolving buffer |
| | and sonicate until SIF powder is completely dissolved. Then |
| | dilute to volume with FaSSIF dissolving buffer and mix well. |
| | The FaSSIF solution can be stored at 4° C. for 7 days and |
| | should be equilibrated for 2 hours to RT before use. |
| | FeSSIF: FeSSIF dissolving buffer: weigh 0.82 mL of glacial |
| | acetic acid, 403.1 mg of sodium hydroxide and 1.188 g of |
| | sodium chloride into a 100-mL volumetric flask. Add |
| | appropriate volume of purified water to dissolve the solids. |
| | Then add sufficient purified water closely to the target |
| | volume and adjust to pH 5.01. Dilute to volume with purified |
| | water, mix well. |
| | Weigh 1120.1 mg of SIF powder into a 100-mL volumetric |
| | flask. Add appropriate volume of FeSSIF dissolving buffer |
| | and sonicate until SIF powder is completely dissolved. Then |
| | dilute to volume with FeSSIF dissolving buffer and mix well. |
| | The FeSSIF solution can be stored at 4° C. for 7 days and |
| | should be equilibrated for 2 hours to RT for before use. |
| | H₂O: Milli-Q water. |
| Proce- | 1. ~20 mg of starting material (822904-01-A) is added to 4 |
| dures | mL of media in a 5-mL glass vial. |
| | 2. Roll the vial at 37 ± 1° C. in an incubator at the rate of 25 |
| | rpm. Sample at 1 hr, 4 hrs, 24 hrs. After centrifugation and |
| | filtration, transfer the filtrate for HPLC and pH test. Check |
| | XRPD of residual solids. |

### Preparation of 6-(3-(4,4-difluoro-4-(2-methoxypyridin-4-yl)butanoyl)-3,8-diazabicyclo[3.2.1]octan-8-yl)nicotinonitrile

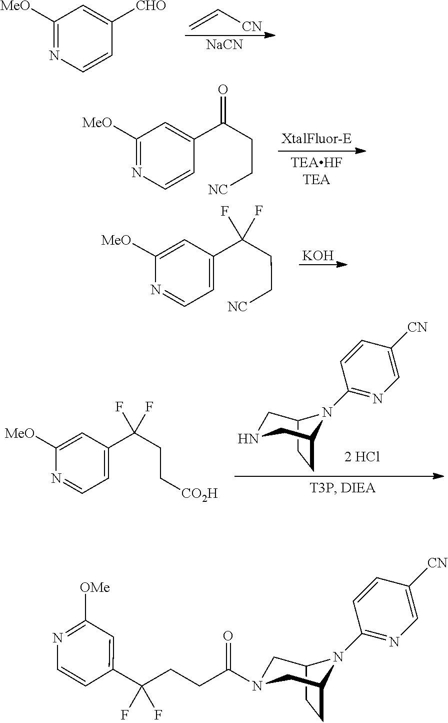

Step 1: In a dried round-bottom equipped with a magnetic stirrer was combined 2-methoxyisonicotinaldehyde (1 equiv, PharmaBlock, Inc) and sodium cyanide (0.25 equiv, Sigma-Aldrich) in MeCN (0.3 M). The resulting suspension was deoxygenated via sub-surface purging with nitrogen for 15 min before acrylonitrile (0.95 equiv, 14 M solution in MeCN, Acros) was added dropwise over 5 min. After 3.5 h of stirring at RT, the reaction was carefully quenched with glacial acetic acid (1 equiv, Sigma-Aldrich), diluted further with water and extracted with EtOAc (2×). The combined organic extracts were washed further with saturated aq. NaHCO₃ and brine, dried over MgSO₄ and filtered. Concentration of the filtrate in vacuo afforded 4-(2-methoxypyridin-4-yl)-4-oxobutanenitrile as a solid (92% yield).

Step 2: In a Nalgene® bottle equipped with a magnetic stirrer was dissolved 4-(2-methoxypyridin-4-yl)-4-oxobutanenitrile (1 equiv) from the previous step in dichloromethane (0.5 M). To this was then added sequentially triethylamine trihydrofluoride (5 equiv, Sigma-Aldrich), triethylamine (2.5 equiv, Sigma-Aldrich) and finally XtalFluor-E® (5 equiv, Sigma-Aldrich). The resulting reaction mixture was stirred under a nitrogen atmosphere at RT for 3 days. The reaction mixture was diluted with dichloromethane and then carefully added into ice. The organic layer was separated, washed sequentially with water, saturated aq. NaHCO₃ and brine, dried over MgSO₄, filtered and the filtrate concentrated in vacuo. Purification of the crude product thus obtained by way of column chromatography (SiO₂, gradient elution: Hex→EtOAc) afforded 4,4-difluoro-4-(2-methoxypyridin-4-yl)butanenitrile as a white solid (72% yield).

Step 3: In a round-bottom flask equipped with a magnetic stirrer was combined 4,4-difluoro-4-(2-methoxypyridin-4-yl)butanenitrile (1 equiv) from the previous step and potassium hydroxide (4 equiv, Alfa Aesar) in a 9:1 (v/v) solution of water and ethanol (0.30 M). The resulting solution was heated at 80° C. for 12 h. The reaction mixture was then cooled to RT and washed with tert-butyl methyl ether. The aqueous layer was separated, treated with activated charcoal and filtered through a pad of celite. The filtrate thus obtained was then cooled to 0° C. and its pH was carefully adjusted to ˜2 with the dropwise addition of 3 M aq. HCl. The title compound was then isolated via vacuum filtration, washed further with water and hexanes, and dried in vacuo until constant weight (80% yield).

Step 4: In a dried round-bottom flask equipped with a magnetic stirrer was combined 4,4-difluoro-4-(2-methoxypyridin-4-yl)butanoic acid (1 equiv) from the previous step and 6-(3,8-diazabicyclo[3.2.1]octan-8-yl)nicotinonitrile bis hydrochloride (1.05 equiv, vide infra) in THF (0.43 M). The resulting reaction mixture was then cooled to 0° C. before DIEA (5 equiv, Sigma-Aldrich) was added dropwise over 30 minutes. After another 30 minutes of stirring between 0° C. and 10° C., propylphosphonic anhydride (1.5 equiv, 50 wt % solution in EtOAc, Sigma-Aldrich) was added dropwise to the reaction mixture over 30 minutes. The resulting yellow solution was allowed to stir at RT for 2 h. The reaction mixture was then poured into water and the organic layer was separated. The aqueous layer was back-extracted with tert-butyl methyl ether. The organic extracts were combined and treated with activated carbon. After 12 h of vigorous stirring at RT, the black suspension was filtered through a bed of celite. The filtrate thus obtained was then concentrated in vacuo and the resulting residue was taken up in warm isopropanol. After 12 h of vigorous stirring at RT, the desired product crystallized out as a white solid. This solid was isolated via vacuum filtration, washed further with cold isopropanol, and dried under vacuum until constant weight (80% yield). LCMS: m/z=428.1 [M+H]⁺; ¹H NMR (DMSO-d₆): δ=8.50 (d, J=2.5 Hz, 1H), 8.29 (d, J=5.0 Hz, 1H), 7.88 (dd, J=9.0, 2.5 Hz, 1H), 7.11 (dd, J=5.0, 1.0 Hz, 1H), 3.93 (s, 1H), 6.91 (d, J=9.0 Hz, 1H), 4.69 (br s, 2H), 4.06 (d, J=12.0 Hz, 1H), 3.88 (s, 3H), 3.62 (d, J=13.0 Hz, 1H), 3.22 (d, J=11.5 Hz, 1H), 2.73 (d, J=12.0 Hz, 1H), 2.61˜2.51 (m, 1H), 2.49˜2.37 (m, 2H), 2.36˜2.28 (m, 1H), 1.94˜1.83 (m, 2H), 1.81˜1.77 (m, 1H), 1.61˜1.53 (m, 1H).

### Preparation of 6-(3,8-diazabicyclo[3.2.1]octan-8-yl)nicotinonitrile bis hydrochloride

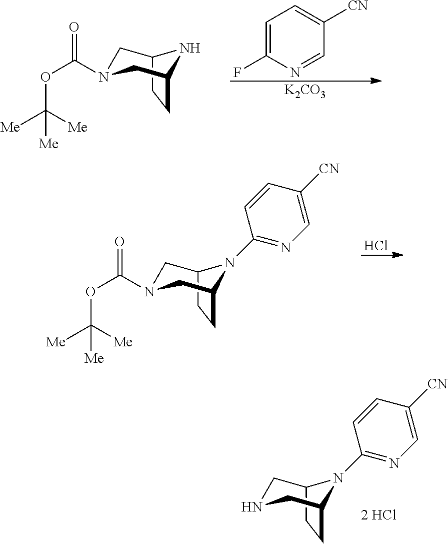

Step 1: In a dried round-bottom flask equipped with a magnetic stirrer and a reflux condenser was dissolved tert-butyl 3,8-diazabicyclo[3.2.1]octane-3-carboxylate (1 equiv, PharmaBlock, Inc.) and 5-cyano-2-fluoropyridine (1.2 equiv, Combi-Blocks) in acetonitrile (0.45 M). To this was then added potassium carbonate (2 equiv, Sigma-Aldrich) in one rapid portion and the resulting suspension was stirred at reflux for 3 h. The reaction suspension was then cooled to RT and filtered. The insoluble was rinsed further with acetonitrile and the filtrate was concentrated in vacuo. The resulting residue was then partitioned between EtOAc and water. The organic layer was separated and washed further with water (2×) and brine. The combined aqueous washes were backextracted with EtOAc (2×). The EtOAc extracts were then combined, dried over MgSO₄, filtered and the filtrate concentrated in vacuo. The resulting oil, which solidified upon standing, was taken up in warm EtOAc and then added an equal volume of hexanes. Upon cooling to RT, slow precipitation of white crystalline solid was observed. This solid impurity was removed via filtration and discarded. The filtrate was then concentrated in vacuo and the crude product thus obtained was purified further by way of column chromatography (SiO₂, gradient elution: Hex→1:1 (v/v) Hex: EtOAc) to afford tert-butyl 8-(5-cyanopyridin-2-yl)-3,8-diazabicyclo[3.2.1]octane-3-carboxylate as a white, crystalline solid (94% yield).

Step 2: In a dried round-bottom flask equipped with a magnetic stirrer was dissolved tert-butyl 8-(5-cyanopyridin-2-yl)-3,8-diazabicyclo[3.2.1]octane-3-carboxylate (1 equiv) from the previous step in dichloromethane (0.42 M). To this was then added at 0° C. HCl (4 equiv, 4 M solution in dioxane, Sigma-Aldrich) in three portions over a period of 30 min. The resulting suspension was stirred at 0° C. for 1 h and then allowed to warm slowly to RT over 16 h. The reaction mixture was added tert-butyl methyl ether and the resulting thick suspension was vigorously stirred at RT for 1 h. Finally, the suspension was filtered, washed further with tert-butyl methyl ether and air-dried to afford the title compound as a white crystalline solid (96% yield).

### Biological Evaluations

### In Vitro Functional Assay of Muscarinic Acetylcholine Receptor Activity

CHO-K1 cells stably expressing human M₁ receptor with aequorin (Perkin Elmer) were grown in F12 media (Gibco) containing 10% FBS (ATCC), 0.4 mg/mL geneticin (Sigma-Aldrich) and 0.25 mg/mL Zeocin (Invitrogen). Cells were grown as per the manufacturer's protocol. For compound testing, cells were grown to confluency and detached gently with Accutase (Sigma-Aldrich) followed by centrifugation for 5 min at 150×g. Cells were then re-suspended in assay buffer (i.e. DMEM/F-12 HEPES without phenol red (Invitrogen) with 0.1% BSA (Sigma-Aldrich)) at a density of 5×10⁶ cells/ml. Under sterile conditions, 5 μM coelenterazine (Invitrogen) was added to the cells, mixed, then incubated at room temperature protected from light, with gentle agitation, for 4 h.

Primary compound plates were prepared in 100% DMSO in opaque 96-well plates (VWR) and serially diluted in half log increments. Secondary compound plates were prepared at 3× concentration in assay buffer. The compound (6-(3-(4,4-difluoro-4-(2-methoxypyridin-4-yl)butanoyl)-3,8-diazabicyclo[3.2.1]octan-8-yl)nicotinonitrile) was added to white, clear bottom tissue culture treated 96-well plates (Fisher Scientific). Coelenterazine-loaded cells were then added at 5×10⁵ cells/well.

The compound and cells were incubated at room temperature for 30 min in the dark. Acetylcholine at the EC₅₀ concentration was added and calcium flux measured using a FlexStation 3 (Molecular Devices). Sigmoidal dose-response curves were generated by measuring luminescence over 40 sec and calculating the area under the curve. Dose response curves and IC₅₀ values were generated using Prism (GraphPad). The compound was tested at a final concentration range of 100 μM to 10 μM in 0.10% DMSO.

CHO-K1 cells stably expressing human M₂, M₃ and M₄ receptors, respectively, with aequorin (Perkin Elmer) were used to assess a test compound's ability to dose-dependently reverse the EC₅₀ acetylcholine response. The IC₅₀ of the compound was calculated from the dose response curve.

Results (IC₅₀) for M₁, M₂, M₃ and M₄ receptors for the compound are shown in Table 16.

TABLE 16

| | M₁ IC₅₀ | M₂ IC₅₀ | M₃ IC₅₀ | M₄ IC₅₀ |
|---|---|---|---|---|
| Compound | A | D | C | C |

A = IC₅₀ of less than 10 nM;
B = IC₅₀ less than 100 nM but greater than or equal to 10 nM;
C = IC₅₀ less than 1 μM (1,000 nM) but greater than or equal to 100 nM;
D = IC50 less than 10 μM (10,000 nM) but greater than or equal to 1 μM (1,000 nM);
E = IC50 greater than 1 μM (1,000 nM)

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A crystalline compound having the formula: 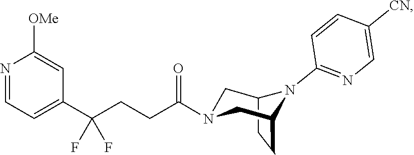
characterized by an x-ray powder diffraction pattern with peaks at 13.4±0.15° 2θ, 14.0±0.15° 2θ, 15.2±0.15° 2θ, 19.3±0.15° 2θ, 20.0±0.15° 20 and 21.2±0.15° 2θ.

2. The crystalline compound of claim 1, wherein the compound is characterized by an x-ray powder diffraction pattern with peaks at 6.7±0.15° 2θ, at 13.4±0.15° 2θ, 14.0±0.15° 20, 15.2±0.15° 2θ, 17.7±0.15° 2θ, 18.1±0.15° 2θ, 18.8±0.15° 2θ, 19.3±0.15° 2θ, 19.8±0.15° 2θ, 20.0±0.15° 2θ, 21.0±0.15° 2θ, 21.2±0.15° 2θ, and 24.1±0.15° 2θ.

3. The crystalline compound of claim 1, wherein the compound is characterized by an x-ray powder diffraction pattern with peaks at 6.7±0.15° 2θ, 10.7±0.15° 2θ, 13.4±0.15° 2θ, 14.0±0.15° 2θ, 15.2±0.15° 2θ, 17.7±0.15° 2θ, 18.1±0.15° 2θ, 18.8±0.15° 2θ, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 27.8±0.15° 2θ, and 30.6±0.15° 2θ.

4. The crystalline compound of claim 1, wherein the compound is characterized by an x-ray powder diffraction pattern with peaks at 6.7±0.15° 2θ, 10.7±0.15° 2θ, 13.4±0.15° 2θ, 14.0±0.15° 2θ, 15.2±0.15° 2θ, 16.6±0.15° 2θ, 17.7±0.15° 2θ, 18.1±0.15° 2θ, 18.4±0.15° 2θ, 18.8±0.15° 2θ, 19.3±0.15° 2θ, 19.8±0.15° 2θ, 20.0±0.15° 2θ, 20.7±0.15° 2θ, 21.0±0.15° 2θ, 21.2±0.15° 2θ, 24.1±0.15° 2θ, 25.9±0.15° 2θ, 26.3±0.15° 2θ, 26.6±0.15° 2θ, 27.0±0.15° 2θ, 27.8±0.15° 2θ, and 30.6±0.15° 2θ.

5. The crystalline compound of claim 1, wherein the compound is further characterized as having a differential scanning calorimetry endotherm onset at about 117° C.

6. A pharmaceutical composition comprising a crystalline compound of claim 1 and at least one pharmaceutically acceptable excipient.

7. A method of treating a neurodegenerative disorder in a subject in need thereof, comprising administering to the subject a therapeutically effective amount of a crystalline compound of claim 1.

8. The method of claim 7, further comprising the administration of one or more immunomodulatory agents.

9. The method of claim 8, wherein the one or more immunomodulatory agents are selected from: an IFN-β 1 molecule; a corticosteroid; a polymer of glutamic acid, lysine, alanine and tyrosine or glatiramer; an antibody or fragment thereof against alpha-4 integrin or natalizumab; an anthracenedione molecule or mitoxantrone; a fingolimod or FTY720 or other S1P1 functional modulator; a dimethyl fumarate or other NRF2 functional modulator; an antibody to the alpha subunit of the IL-2 receptor of T cells (CD25) or daclizumab; an antibody against CD52 or alemtuzumab; an antibody against CD20 or ocrelizumab; and an inhibitor of a dihydroorotate dehydrogenase or teriflunomide.

10. A method of treating a demyelinating disease in a subject in need thereof, comprising administering to the subject a therapeutically effective amount of a crystalline compound of claim 1.

11. The method of claim 10, wherein the demyelinating disease is a demyelinating disease of the central nervous system.

12. The method of claim 11, wherein the demyelinating disease is multiple sclerosis.

13. The method of claim 10, wherein the demyelinating disease is a demyelinating disease of the peripheral nervous system.

14. The method of claim 10, further comprising the administration of one or more immunomodulatory agents.

15. The method of claim 14, wherein the one or more immunomodulatory agents are selected from: an IFN-β 1 molecule; a corticosteroid; a polymer of glutamic acid, lysine, alanine and tyrosine or glatiramer; an antibody or fragment thereof against alpha-4 integrin or natalizumab; an anthracenedione molecule or mitoxantrone; a fingolimod or FTY720 or other S1P1 functional modulator; a dimethyl fumarate or other NRF2 functional modulator; an antibody to the alpha subunit of the IL-2 receptor of T cells (CD25) or daclizumab; an antibody against CD52 or alemtuzumab; an antibody against CD20 or ocrelizumab; and an inhibitor of a dihydroorotate dehydrogenase or teriflunomide.

16. A method of treating a neuropathic disease, optionally a peripheral neuropathy, in a subject in need thereof, comprising administering to the subject a therapeutically effective amount of a crystalline compound of claim 1.

17. The method of claim 16, wherein the neuropathic disease is diabetic neuropathy.

18. The method of claim 16, further comprising the administration of one or more immunomodulatory agents.

19. The method of claim 18, wherein the one or more immunomodulatory agents are selected from: an IFN-β 1 molecule; a corticosteroid; a polymer of glutamic acid, lysine, alanine and tyrosine or glatiramer; an antibody or fragment thereof against alpha-4 integrin or natalizumab; an anthracenedione molecule or mitoxantrone; a fingolimod or FTY720 or other S1P1 functional modulator; a dimethyl fumarate or other NRF2 functional modulator; an antibody to the alpha subunit of the IL-2 receptor of T cells (CD25) or daclizumab; an antibody against CD52 or alemtuzumab; an antibody against CD20 or ocrelizumab; and an inhibitor of a dihydroorotate dehydrogenase or teriflunomide.

20. A method of modulating muscarinic acetylcholine receptor M₁ activity in a subject comprising administering to the subject a crystalline compound of claim 1.

21. The method of claim 20, wherein the compound acts as a selective M₁ antagonist.
